(19) United States
(12) Patent Application Publication
(10) Pub. No.: US 2025/0065000 A1
(43) Pub. Date: Feb. 27, 2025

(54) UV Light and Hydrogen Peroxide for Sterilization

(71) Applicant: Z1 Enterprises, Inc., Incline Village, NV (US)

(72) Inventors: Brad Buchanan, Eastsound, WA (US); James Zuegel, Incline Village, NV (US)

(21) Appl. No.: 18/632,689

(22) Filed: Apr. 11, 2024

Related U.S. Application Data
(63) Continuation-in-part of application No. PCT/US2023/086115, filed on Dec. 27, 2023.
(60) Provisional application No. 63/435,468, filed on Dec. 27, 2022.

(51) Int. Cl. A61L 2/10; A61L 2/18; A61L 2/20

(52) U.S. Cl. CPC A61L 2/10; A61L 2/186; A61L 2/208; A61L 2202/11; A61L 2202/122; A61L 2202/23

(57) ABSTRACT

An exemplary method for sterilizing at least one item may include exposing the at least one item to a hydrogen peroxide environment for an exposure time of sufficient duration to sterilize the at least one item; separating the at least one item from the hydrogen peroxide environment; and illuminating the at least one item with ultraviolet light to decompose remnant hydrogen peroxide to water and oxygen. The at least one item may be a Primary Container Closure System (PCCS), where the method further includes filling the sterilized at least one PCCS with parenteral product after the illuminating.

### CROSS-REFERENCE TO RELATED APPLICATIONS

This application is a bypass continuation-in-part of PCT application PCT/US2023/086115, filed on Dec. 27, 2023, which in turn claims the benefit of priority to U.S. Provisional Patent Application Ser. No. 63/435,468, filed Dec. 27, 2022, each of which is incorporated by reference in its entirety herein.

### FIELD OF THE INVENTION

The invention generally relates to sterilization, and more particularly to sterilization of containers for medication and of medical devices.

### BACKGROUND

Sterilization of medical devices, and containers that hold parenteral drugs or other drug products which are sterile or require biological decontamination, is important for preventing infection in patients that receive those medical devices, drugs, or other products susceptible to microbial spoilage. Generally, the requirements for sterility, freedom from pyrogens, freedom from particulate matter, and freedom from other contaminants are similar across different medical devices and for containers that hold drugs. Such requirements are similar, and important, for other products susceptible to microbial spoilage, including food, beverage, and cosmetic products. Additionally, processes for bio-decontamination of aseptic processing environments have similar requirements.

Although there are similarities in the requirements described above, parenteral drug products pose the most challenging requirements. As such, parenteral products may be considered a useful “worst case” for demonstrating the usefulness of a sterilization technique. Consequently, parenteral products are the focus of this background section. The term “parenteral” means “taken into the body or administered in a manner other than through the digestive tract,” such as by intravenous administration or an injection. Parenteral products may be drugs, may be solutions including saline or Ringer's lactate, or other fluids useful in medicinal treatment of humans and/or other organisms. Such parenteral products may be introduced directly into the bloodstream, into muscle, into an internal organ, into a wound, or into another part of the body.

Production of parenteral products is rigorously guided by the FDA and applicable U.S. Pharmacopeia (USP) standards. The basic requirements for parenteral products are: (a) packaging of parenteral products must be sterile upon completion of the manufacturing process; (b) materials, manufacturing processes and sterilization must not affect or change packaged product; and (c) the closure and sealing system used in the process must insure maintenance of sterility while not contaminating the product.

The most common forms of parenteral packaging are: (a) ampoules (glass); (b) vials (glass and plastic); (c) pre-filled syringes (glass and plastic); (d) cartridges (glass); (c) bottles (glass and plastic); and (f) bags (plastic). Parenteral packages are commonly referred to in the industry as Primary Container Closure Systems (PCCSs). Approximately 50% of small volume injectable parenteral products are packaged using vials, with syringes accounting for another 30%. Parenteral products are generally sterilized in one of two ways: (a) bulk sterilization of the packaged parenteral product, or (b) localized package sterilization combined with sterile filtering and aseptic Fill and Seal (F&S). Wherever possible, parenteral manufacturers fill the packaging with the drug/solution, seal the packages, and transfer them to a bulk sterilization facility where they are sterilized such as by exposure to steam or radiation. Bulk sterilization provides greater assurance of sterilization of the finished product than aseptic processing methods. However, a significant number of parenteral products cannot be bulk sterilized, because those products contain pharmaceutical components which cannot tolerate the heat or radiation applied during bulk sterilization; the only way to produce those products is by using aseptic processing methods.

Sterility is defined as the complete absence of microbial life; such microbial life may be referred to as “bioburden.” There are four main methods that sterilize items used in parenteral manufacturing: (a) heat (bulk); (b) gas (bulk); (c) radiation (bulk and localized); and (d) filtration (localized).

Heat, ethylene oxide (EtO) exposure, and gamma radiation each have been and continue to be used for bulk sterilization. However, as noted above, a significant number of PCCSs cannot be bulk sterilized, and each of those three sterilization methods have significant drawbacks that make them unsuitable for use with those PCCSs. In recent years, vapor-phase hydrogen peroxide (VHP) has been introduced for sterilization of PCCSs and medical devices. Hydrogen peroxide works by producing destructive hydroxyl free radicals that can attack membrane lipids, DNA, and other essential cell components. However, VHP requires pressure-to-vacuum cycling, which takes significant time, in order to ensure that substantially all of the vapor-phase hydrogen peroxide, as well as liquid-phase hydrogen peroxide that has condensed in the PCCSs, has been removed from those PCCSs after they have been sterilized and before they have been filled with parenteral product. The time to sterilize PCCSs with VHP can be up to eight hours, or more, which is undesirably long in a high-throughput manufacturing process. Where a facility runs a single shift, an eight-hour process consumes the entire workday, meaning that the effective process time using VHP is twenty-four hours: the PCCSs sterilized on one workday cannot then be used until the start of the next workday. VHP sterilization is typically performed in three phases: (1) preconditioning (pull vacuum & remove humidity from chamber holding product); (2) sterilization (35% H2O2 is vaporized and injected in pulses into the chamber); and (3) aeration (apply vacuum to chamber and H2O2 removed by injecting air and/or steam). In general, preconditioning takes 1-2 hours, sterilization takes 1-2 hours, and aeration takes 2-5 hours. Aeration is the longest phase, because it is important to remove virtually all residual hydrogen peroxide from the sterile PCCSs. Steps must be taken to limit exposure of the parenteral product to residual hydrogen peroxide in the PCCSs, which as a highly reactive oxidizing agent can have adverse effects on product quality if not controlled. This particularly holds true for protein therapeutics, such as monoclonal antibodies. Repeated pressure-to-vacuum cycling in the aeration phase removes virtually all residual hydrogen peroxide from the PCCSs, but at the cost of consuming more time than any other phase in the VHP sterilization process.

A significant challenge with the manufacture of finished parenteral medication is the sterilization of PCCSs that hold that medication. In many instances, those PCCSs are sterilized off-site at a service provider that specializes in sterilization. However, after such sterilization, the PCCSs must be transferred into an aseptic ISO Class 5 space for aseptic filling with medication. This transfer can be complex, time-consuming, and subject to error, which can cause contamination and loss of entire batches of product as a result. Autoclaving with pressurized steam may be used to kill microbes and partially depyrogenate proteins. However, the use of autoclaving can require multi-layered packaging that generates substantial waste, requires significant equipment and energy to operate that equipment, requires significant cool-down time and space, and requires transfer into an ISO Class 5 space for aseptic filling with medication. Dry heat tunnels may be used to sterilize PCCSs before filling by thermally destroying microbes and pyrogens. However, the use of dry heat tunnels also requires significant equipment and energy to operate that equipment, and requires significant cool-down time and space. The cooling process may occur within ISO Class 5 space; however, the expense and complexity of maintaining an ISO Class 5 space increases with the size of that space, and a cool-down location for PCCSs in that ISO Class 5 space increases the required size of that space.

Filters with sub-micron pore size are used to remove microbial contamination from liquid products. In appropriate aseptic filling processes, filtration can be used with previously sterilized packaging to produce sterile parenteral products. However, while filters can remove bioburden from a liquid drug or solution, they cannot sterilize the container into which that liquid drug or solution is placed.

As described in the priority documents, PCT application PCT/US2023/086115 and U.S. provisional patent application Ser. No. 63/435,468, devices and methods for utilizing pulsed UV light are efficient, cost effective, and have verifiable efficacy. However, prior to sterilization and filling, parenteral packaging may be stored in a warehouse or other location for a substantial period of time. Parenteral packaging (i.e., PCCSs) are typically provided pre-cleaned and pre-sterilized in bulk packaging, so they are not exposed to warehouse conditions. The bulk packaging material is typically multi-layered, utilizing at least two, and often three, layers of sterile barriers. However, multi-layer packaging is expensive and environmentally unfriendly. But, any residual bioburden and other biological material remaining in the cleaned and sterilized PCCSs still contaminates that unfilled parenteral packaging. Further, elaborate equipment and processes are required to move the PCCSs into an aseptic processing environment without introducing contaminants. Thus, there is a need to reduce that contamination of the parenteral packaging prior to sterilization with pulsed UV light, which would make that sterilization even more effective.

### SUMMARY OF THE INVENTION

In an aspect of the invention, pulsed UV light is used in conjunction with liquid or vapor phase hydrogen peroxide sterilization of items. Those items are first sterilized with hydrogen peroxide, and then exposed to pulsed UV light. That pulsed UV light neutralizes any remnant hydrogen peroxide in those items, turning it to water and gaseous oxygen. As a result, one of the most time-consuming parts of hydrogen peroxide sterilization—the removal of all hydrogen peroxide from the items—can be eliminated, improving throughput of a high-volume manufacturing process.

According to some embodiments, a method for sterilizing at least one item may include exposing the at least one item to a hydrogen peroxide environment for an exposure time of sufficient duration to sterilize the at least one item; separating the at least one item from the hydrogen peroxide environment; and illuminating the at least one item with ultraviolet light to decompose remnant hydrogen peroxide to water and oxygen. The at least one item may be a Primary Container Closure System (PCCS), where the method further includes filling the sterilized at least one PCCS with parenteral product after the illuminating.

According to some embodiments, a method for sterilizing workpiece items may include providing a sterilization cabinet including a sterilization chamber, at least one portal to the sterilization chamber openable to load at least one workpiece item into the sterilization chamber, a carrier movable within the sterilization chamber, and at least one UV light source that generates pulsed UV light pulses directed into the sterilization chamber; exposing at least one workpiece item to hydrogen peroxide; after the exposing, loading at least one workpiece item onto a carrier in the sterilization chamber, the carrier in a loading position; moving the carrier to move at least one workpiece item to a sterilization position within the sterilization chamber; sterilizing each workpiece item by applying pulsed UV light thereto; moving the carrier to an unloading position; and causing each workpiece item to exit the sterilization chamber.

According to some embodiments, a method for sterilizing at least one workpiece item may include: providing a sterilization chamber configured for hydrogen peroxide sterilization and a UV light source configured to illuminate the interior of the sterilization chamber; loading the at least one workpiece item into the sterilization chamber; contacting the at least one workpiece item with hydrogen peroxide; separating the at least one workpiece item from the hydrogen peroxide, leaving remnant hydrogen peroxide on the at least one workpiece item; and illuminating the at least one workpiece item with UV light, wherein the illuminating decomposes remnant hydrogen peroxide on the at least one workpiece item.

### BRIEF DESCRIPTION OF THE DRAWINGS

FIG. 1 is a perspective view of a sterilization cabinet including a sterilization chamber.

FIG. 2 is a front perspective view of the sterilization chamber of FIG. 1.

FIG. 3 is a closer front perspective view of the sterilization chamber of FIG. 1.

FIG. 4 is a top schematic view of the sterilization chamber of FIG. 1.

FIG. 5 is a perspective view of an exemplary nest holding a PCCS, where the nest is in an open configuration.

FIG. 6 is a perspective view of the nest and PCCS of FIG. 5, where the nest is in a closed configuration.

FIG. 7 is a side view of the nest and PCCS of FIG. 6, where the nest is in the closed configuration.

FIG. 8 is a schematic view of the sterilization cabinet of FIG. 1 illustrating the electrical and data interconnection of components therein.

FIG. 9 is a flowchart illustrating a method of operating the sterilization cabinet of FIG. 1.

FIG. 10 is a schematic view of the sterilization chamber of FIG. 1 located in a clean room.

FIG. 11 is a schematic view of a first environment in which the sterilization cabinet of FIG. 1 is used.

FIG. 12 is a flowchart illustrating a method of operating the sterilization cabinet in the first environment of FIG. 11.

FIG. 13 is a schematic view of a second environment in which the sterilization cabinet of FIG. 1 is used.

FIG. 14 is a flowchart illustrating a method of operating the sterilization cabinet utilizing an entry tunnel.

FIG. 15 is a flowchart illustrating a method of operating the sterilization cabinet utilizing an exit tunnel.

FIG. 16 is a schematic view of optical elements that manage UV light inside the sterilization chamber.

FIG. 17 is a schematic view of a sterilization chamber used in conjunction with an entry tunnel and an exit tunnel.

FIG. 18A is a schematic view of exemplary serial operation of a sterilization chamber used in conjunction with an entry tunnel and an exit tunnel.

FIG. 18B is a schematic view of exemplary semi-parallel operation of a sterilization chamber used in conjunction with an entry tunnel and an exit tunnel.

FIG. 18C is a schematic view of exemplary parallel operation of a sterilization chamber used in conjunction with an entry tunnel and an exit tunnel.

FIG. 19 is a schematic view of the first environment of FIG. 11 in which the sterilization cabinet of FIG. 1 is used, further showing exemplary entry and exit tunnels associated with the sterilization cabinet.

FIG. 20 is a flowchart illustrating the process of FIG. 9, with additional steps utilizing hydrogen peroxide.

FIG. 21 is a flowchart illustrating an exemplary method of sterilizing items using a combination of UV light and hydrogen peroxide.

FIG. 22 is a schematic view of a vapor phase hydrogen peroxide sterilization chamber utilizing a UV light source.

The use of the same reference symbols in different figures indicates similar or identical items.

### DETAILED DESCRIPTION

### Sterilization Cabinet

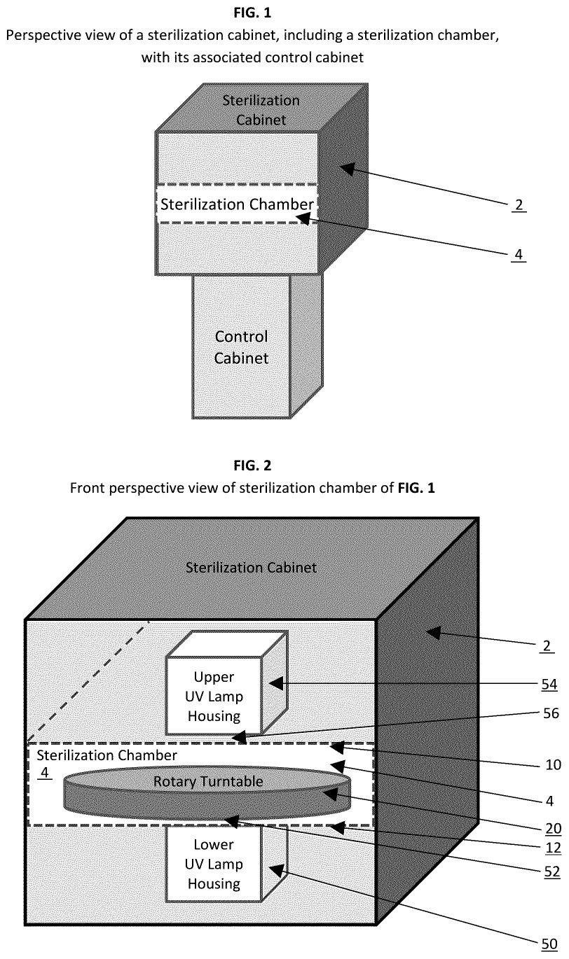

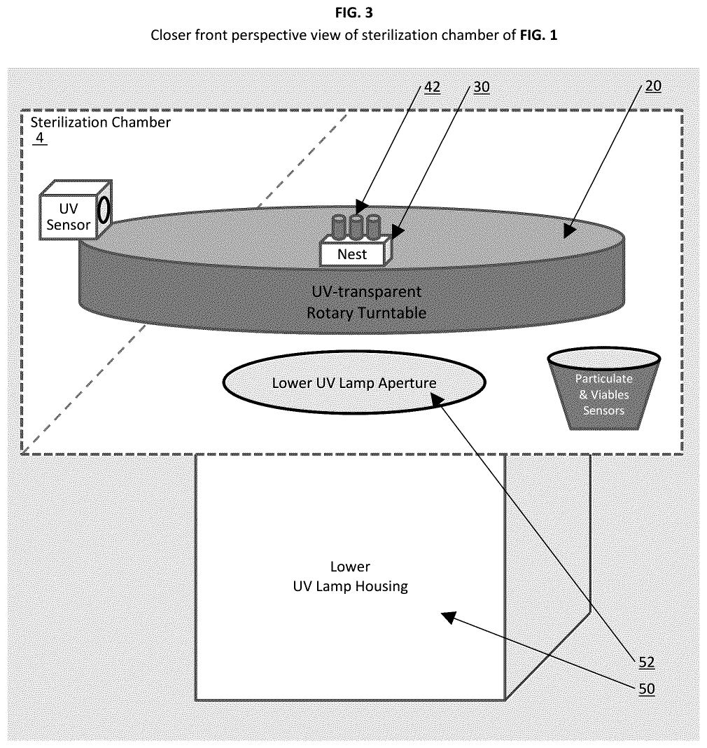

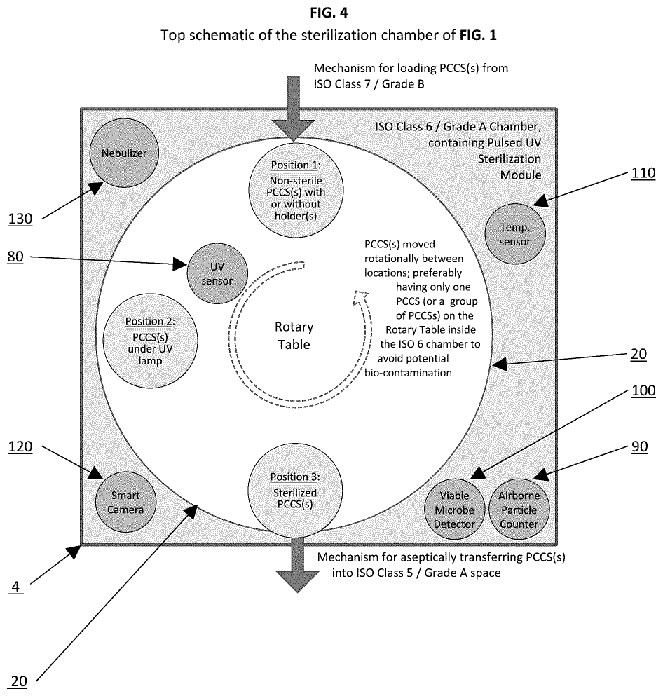

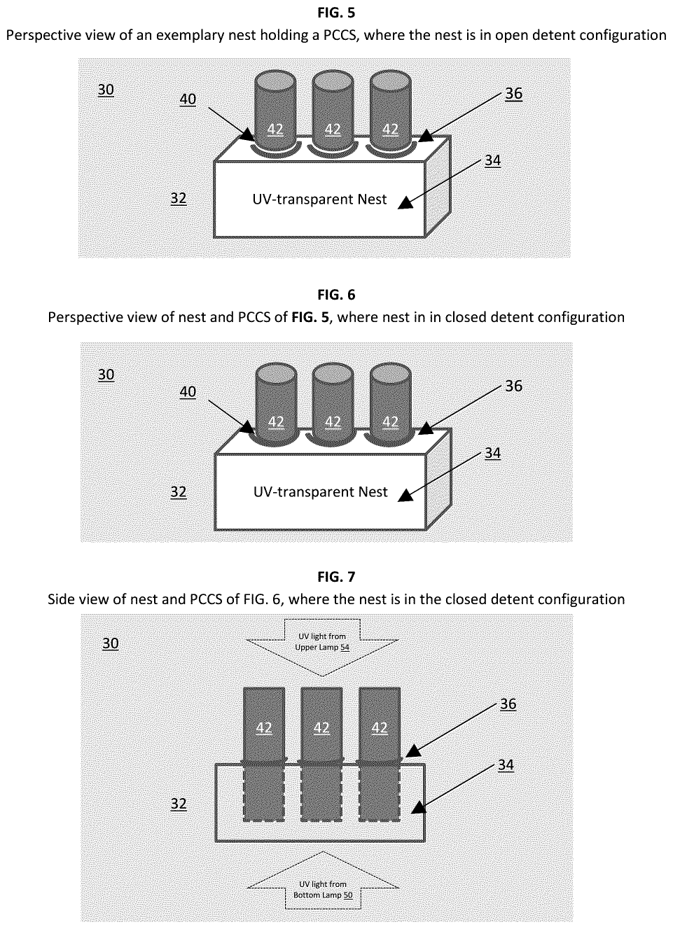

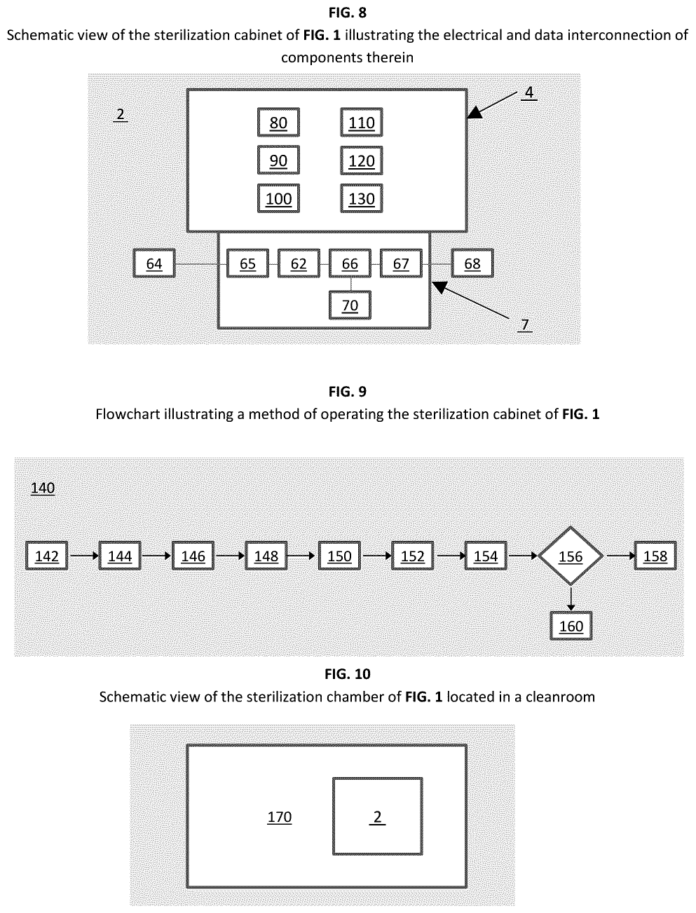

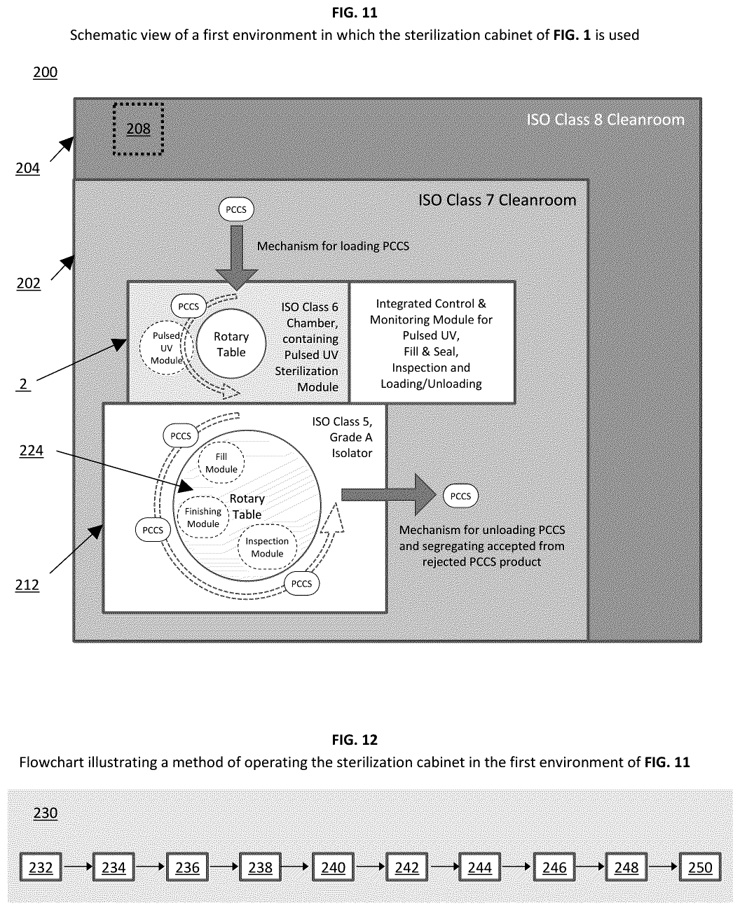

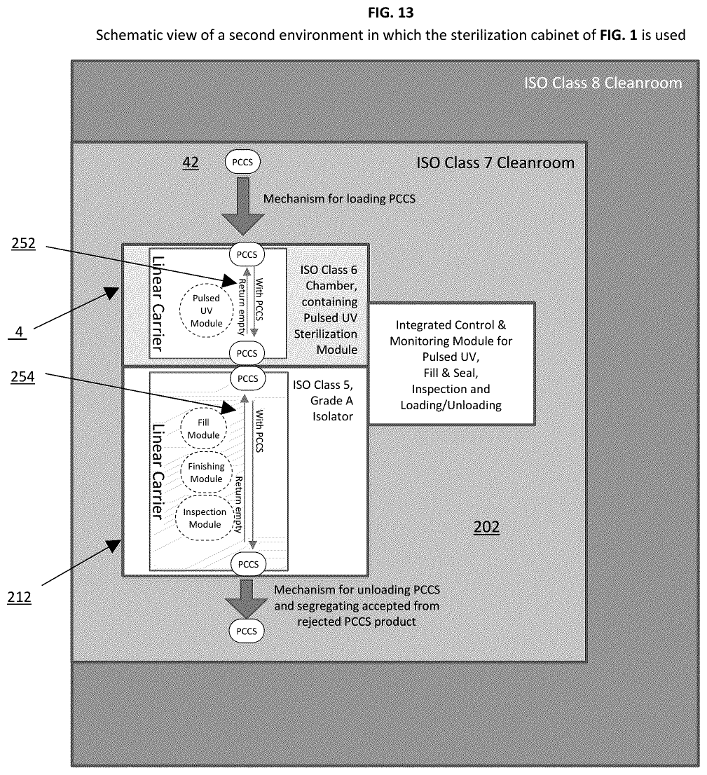

Referring to FIGS. 1-4, a sterilization cabinet 2 is shown. A sterilization chamber 4 is a space located within the sterilization cabinet 2. The sterilization chamber 4 may have any suitable size and shape that allows for the sterilization of one or more items at a time. According to some embodiments, for simplicity of fabrication, the sterilization chamber 4 may be generally box-shaped. For example, three of the lateral walls bounding the sterilization chamber 4 may be walls of the sterilization cabinet; the fourth lateral wall bounding the sterilization chamber 4 may be a door, and the upper and lower surfaces 10, 12 of the sterilization chamber 4 may be generally flat surfaces with apertures 52 defined therethrough. The door may be hinged relative to a remainder of the sterilization cabinet 2, and may rotate about that hinge to allow access to the sterilization chamber 4 for loading and unloading items to be sterilized, and to block access to the sterilization chamber during sterilization. Alternately, the door may be slidable, foldable, or otherwise movable relative to the sterilization chamber 4 to alternately allow access and block access thereto as described above

A carrier is located within the sterilization chamber 4. The carrier may hold one or more nests 30 and/or PCCSs 42, as described in greater detail below, and positions and/or orients those nests and/or PCCSs during sterilization. As one example, the carrier may be a rotary table 20. Alternately, the carrier may be a linear stage, conveyor belt, roller conveyor, or any other structure or mechanism capable of holding one or more nests and/or PCCSs during sterilization. A rotary table is used in subsequent description to demonstrate the underlying concepts for these other structures or mechanisms. According to some embodiments, the carrier is not removable from the sterilization chamber 4 during normal use, although the carrier may be removable from the sterilization chamber 4 for maintenance or replacement. According to other embodiments, the carrier may be removable from the sterilization chamber 4 for loading and unloading one or more nests and/or PCCSs therefrom. According to other embodiments, the carrier may be configured to hold one or more different, or additional, items to be sterilized; a nest may be used to hold such items, a different holder may be used to hold such items, or a nest or separate holder may be omitted for such items.

Where the carrier is a rotary table 20, the rotary table 20 may be substantially UV-transparent. As used in this document, the term “substantially UV-transparent” means that at least 95% of UV light that is received by a structure passes through that structure. The rotary table 20 may be partially UV-transparent, as long as sufficient UV light penetrates the rotary table 20 to reach the one or more nests and/or PCCSs placed thereon to achieve sterilization. According to some embodiments, the rotary table 20 may be fabricated from quartz glass. Quartz glass is substantially UV-transparent, and is strong enough to be suitable for use as a rotary table 20. According to other embodiments, the rotary table 20 may be fabricated from fused silica and/or any other suitable material. The rotary table 20 may be fabricated from a combination of two or more different UV-transparent materials. According to other embodiments, the rotary table 20 may be UV-translucent rather than substantially UV-transparent. As used in this document, the term “UV-translucent” means that less than 95% of UV light that is received by a structure passes through that structure. Where the rotary table 20 is UV-translucent, the intensity, power or other characteristic of UV light that is shone through the rotary table 20 may be increased to compensate for the reduction in the amount of UV light that can pass through the rotary table 20.

The rotary table 20 may be mounted on an axle extending upward through the lower surface 12 of the sterilization chamber 4. The axle may both support and actively rotate the rotary table 20. The axle may be coupled to one or more motors, directly or indirectly, inside or outside the sterilization chamber 4, that actively rotate the rotary table 20. For example, a single motor may be positioned under the lower surface 12 of the sterilization chamber 4, and the axle may be affixed to that motor and extend through an aperture in the lower surface 12 into the sterilization chamber 4. That motor applies rotary power to the axle and thus to the rotary table 20. According to other embodiments, the axle may support the rotary table 20 and passively allow rotation of the rotary table 20. In such embodiments, the rotary table 20 may be rotated such as by the use of one or more motor coupled to an edge of the rotary table 20, such as by rollers, and the rotary table 20 simply rotates around the axle, which does not provide rotary power to the rotary table 20. According to other embodiments, the axle extends downward from the upper surface 10 of the sterilization chamber 4. According to other embodiments, the axle extends through both the upper and lower surfaces 10, 12 of the sterilization chamber 4. The axle may be positioned substantially at the lateral and longitudinal center of the sterilization chamber 4. The “lateral and longitudinal center” of the sterilization chamber 4 is a point substantially equidistant between the left and right walls 6 of the sterilization chamber 4, and substantially equidistant between the back wall 6 and the inner surface of the door 8. According to other embodiments, the axle may be offset from the lateral and longitudinal center of the sterilization chamber 4. According to other embodiments, a magnetic coupling may be used to connect the motor and the rotary table 20. In such embodiments, the axle may be omitted, and the motor includes part of a magnetic coupling and the rotary table 20 includes a corresponding part of a magnetic coupling, such that rotation by the motor of part of the magnetic coupling causes the part of the magnetic coupling associated with the rotary table 20 to rotate, thus causing the rotary table 20 to rotate. The part of the magnetic coupling associated with the motor may be a magnet, and the part of the magnetic coupling associated with the rotary table 20 may be a ferrous or other material that is engaged by the magnet. According to other embodiments, the magnet may be associated with the rotary table 20 and a ferrous or other material is associated with the motor. According to other embodiments, a magnet is associated with each of the rotary table 20 and the motor. According to other embodiments, the axle may be utilized, and a magnet or a material susceptible to magnetism is placed at or near the end of the axle closest to the rotary table 20. The use of magnetic coupling to drive the rotary table 20 reduces the number of apertures that need to be made into the sterilization chamber 4.

Referring also to FIGS. 5-7, a nest 30 is shown. The nest 30 is substantially transparent to UV light. The nest 30 is shaped to hold a particular PCCS 42 to be sterilized. The entirety of the nest 30 advantageously is substantially transparent to UV light, to ensure that all surfaces of the PCCS 42 are exposed to UV light during the sterilization process. According to some embodiments, the body 32 may be simply a lower holder 34 into which a PCCS 42 is placed. According to other embodiments, the body 32 includes an detent feature 36 placed on top of the lower holder 34, with the PCCS 42 positioned between the lower holder 34 and the detent feature 36. In such embodiments, the detent feature 36 may be connected to the lower holder 34 by a hinge or hinges 38. Alternately, the detent feature 36 may be a separate component that is placed on top of the lower holder 34, and that may rest on the lower holder 34 or affirmatively connect to the lower holder 34. The use of both an detent feature 36 and lower holder 34 secures the PCCS 42 in place, and also may ensure that a PCCS 42 that is at least partially flexible is held in a predetermined position to ensure that the entirety of the PCCS 42 is held in an orientation that ensures the entire PCCS 42 receives an amount of UV light to ensure its sterilization. The lower holder 34 may include one or more cradle features 40 that hold and stabilize the PCCS 42 placed in the lower holder 34. As with the other components of the nest 30, the cradle feature or features 40 are substantially transparent to UV light. The nest 30 may be configured to hold a single PCCS 42 at a time, or may be configured to hold two or more PCCSs 42 at once. The nest 30 may be configured to hold a single type of PCCS 42, or may be configured to hold more than one type of PCCS 42, such that the number of different types of nest 30 that are utilized is reduced. For example, the nest 30 may be configured to hold one PCCS 42 that is a vial, and alternately to hold a PCCS 42 that is a syringe. The nest 30 may be fabricated in any suitable manner, such as by injection molding.

Referring to FIGS. 1-4, a lower UV light source 50 is positioned under the lower surface 12 of the sterilization chamber 4, and an upper UV light source 54 is positioned above the upper surface 10 of the sterilization chamber 4. An aperture 52 through the lower surface 12 may allow UV light from the lower UV light source 50 to pass into the sterilization chamber 4, and an aperture 56 through the upper surface 10 may allow UV light from the upper UV light source 54 to pass into the sterilization chamber 4. According to other embodiments, at least one of the lower UV light source 50 and upper UV light source 54 may be positioned within the sterilization chamber 4, such that one or both of the apertures 52, 56 are not needed. According to other embodiments, two or more lower UV light sources 50, and/or two or more upper UV light source 54, are provided, with corresponding apertures. According to some embodiments, the lower UV light source 50 is substantially vertically aligned with the upper UV light source 54. According to other embodiments, the lower UV light source 50 may be vertically offset from the upper UV light source 54. According to some embodiments, the lower UV light source 50 is oriented such that it emits UV light substantially vertically upward, and the upper UV light source 54 is oriented such that it emits UV light substantially vertically downward. According to other embodiments, one or both UV light sources 50, 54 may be oriented at an angle to vertical and emit UV light at an angle relative to vertical.

According to some embodiments, each UV light source 50, 54 is a pulsed xenon lamp. A pulsed xenon lamp may be an arc flashlamp within an enclosure, where xenon is used for excitation inside the enclosure. Other types of lamps that are capable of emitting pulsed UV light may be used. According to some embodiments, one or both UV light sources 50, 54 may be a different kind of pulsed UV-emitting lamp or lamps, one or more UV light-emitting diodes, one or more UV-emitting laser diodes, one or more UV-emitting lasers, or other source of pulsed UV light. According to some embodiments, the lower UV light source 50 may be a different kind of UV light source than the upper light source 54. Regardless of the specific origin of the light emitted by the UV light sources 50, 54, the emitted UV light has the characteristics described below. According to some embodiments, the UV light sources 50, 54 are offset from the lateral and longitudinal center of the sterilization chamber 4, and offset from the axle. According to other embodiments, the UV light sources 50, 54 are positioned substantially at the lateral and longitudinal center of the sterilization chamber 4, and the axle is offset from the lateral and longitudinal center of the sterilization chamber 4.

According to other embodiments, at least one UV light source 50, 54 is a continuous UV light source. Where continuous UV light is used, the time for sterilization of an object in the sterilization chamber 4 may be longer than with the use of a pulsed UV light source. Where at least one UV light source 50, 54 is used, the overall power output by the UV light generated by the UV light source 50, 54 (such as in joules/cm²) is substantially the same as the power emitted by a pulsed UV light source 50, 54. This may be ensured by a higher power continuous UV light source 50, 54, by increasing the dwell time in the sterilization chamber of the item to be sterilized, or by any other suitable method.

According to some embodiments, each UV light source 50, 54 emits electromagnetic radiation between substantially 254-265 nm, in a range of ultraviolet light that may be characterized as deep ultraviolet (DUV) light. DUV light in that range is highly energetic, and thus able to transfer energy to bacteria, viruses, and/or other bioburden at a rate that kills such bacteria, viruses and/or other bioburden. DUV light in that range also has an advantage over shorter wavelengths of UV light under 200 nm (commonly referred to as vacuum ultraviolet (VUV)), which are absorbed by molecular oxygen in the atmosphere and thus require the use of vacuum chambers, adding expense and complexity. Further, DUV light in that range has an advantage over shorter wavelengths of UV light in the extreme ultraviolet (EUV) range having a wavelength of 10 nm to approximately 120 nm, because such wavelengths approach X-ray wavelengths and accordingly the use of such wavelengths requires protection for people in the vicinity, of the same kind as required for the use of X-rays. As used in this document, the term “substantially” as used to modify a single wavelength or a wavelength range refers to small variations in measurement of that single wavelength or wavelength range, which are normal and inescapable parts of measuring a wavelength of electromagnetic energy, as well as normal variations in the emission of electromagnetic energy of a single wavelength or a wavelength range that are normal in the course of operation of a UV light source 50, 54.

According to other embodiments, other ranges of UV light in the DUV portion of the electromagnetic spectrum may be emitted by at least one of the UV light sources 50, 54. For example, the lower end of the range of UV light emitted by at least one of the UV light sources 50, 54 may be 170 nm. As another example, the upper end of the range of UV light emitted by at least one of the UV light sources may be 400 nm. Examples of ranges of wavelengths emitted by one or both UV light sources include, and are not limited to, substantially 254-265 nm, substantially 250-270 nm, substantially 170-265 nm, substantially 180-265 nm, substantially 190-265 nm, substantially 200-265 nm, substantially 210-265 nm, substantially 220-265 nm, substantially 230-265 nm, substantially 240-265 nm, substantially 250-265 nm, substantially 254-275 nm, substantially 254-285 nm, substantially 254-295 nm, substantially 254-305 nm, substantially 254-315 nm, substantially 254-325 nm, substantially 254-335 nm, substantially 254-345 nm, substantially 254-355 nm, substantially 254-365 nm, substantially 254-375 nm, substantially 254-385 nm, substantially 254-395 nm, substantially 254-400 nm, substantially 170-270 nm, substantially 180-270 nm, substantially 190-270 nm, substantially 200-270 nm, substantially 210-270 nm, substantially 220-270 nm, substantially 230-270 nm, substantially 240-270 nm, substantially 250-280 nm, substantially 250-290 nm, substantially 250-300 nm, substantially 250-310 nm, substantially 250-320 nm, substantially 250-330 nm, substantially 250-340 nm, substantially 250-350 nm, substantially 250-360 nm, substantially 250-370 nm, substantially 250-380 nm, substantially 250-390 nm, and substantially 250-400 nm. At least one of the UV light sources 50, 54 may emit light that includes a component of light extending into the visible range of the electromagnetic spectrum, which range is generally considered to be 380-700 nanometers. Emission of light in the visible range does not contribute to sterilization within the sterilization chamber 4, but may be a byproduct of the generation of UV light by at least one UV light source, and such emission of visible light does not affect the sterilization process.

According to some embodiments, the peak emission wavelength of at least one of the UV light sources 50, 54 may be one of 222 nm, 254 nm, 265 nm, 273 nm, and 280 nm. The pulse length, numbers of pulses utilized, energy per pulse (measured in joules/pulse) and other characteristics of the pulsed UV light may be selected by a user to deliver UV light pulses of sufficient energy, wavelength and other characteristics to ensure sterilization of items illuminated by that pulsed UV light.

Referring also to FIG. 8,, the sterilization cabinet 2 may include a control chamber 7 in addition to the sterilization chamber 4 described above. The control chamber 7 may include one or more items useful in the control and monitoring of conditions inside the sterilization chamber 4, as described below. According to other embodiments, the control chamber 7 may be omitted, and the items that otherwise would be included in the control chamber 7 may be positioned outside the sterilization cabinet 2, with electrical and data connections to items inside the sterilization chamber 4 in the same manner as described below. The control chamber 7 may be positioned under the sterilization chamber 4 for ergonomic reasons. Such placement elevates the sterilization chamber 4 for convenient access by a standing person. However, according to other embodiments, the control chamber 7 may be above the sterilization chamber 4, to one or both sides of the sterilization chamber 4, behind the sterilization chamber 4, or in any other suitable location relative to the sterilization chamber 4.

A power supply 62 may be located in the control chamber 7. The power supply 62 is connected to an external source of power. According to some embodiments, the power supply 62 may be connected via a wire or wires 65 to a plug 64 configured to be plugged into a standard wall-mounted power outlet. The power supply 62 may be configured to receive standard AC power such as 110 VAC, 220 VAC, or both. The power supply 62 also may be configured to receive three-phase power and/or other voltages. The power supply 62 may convert AC power received through the plug 64 into DC power at a voltage, amperage and wattage suitable for use by equipment within the sterilization cabinet. According to some embodiments, the power supply 62 is also configured to pass AC power received through the plug 64 to one or more components within the sterilization cabinet 2, such as the UV light sources 50, 54. According to other embodiments, the wire or wires 65 branch between the plug 64 and the power supply 62, or connect to a splitter or other device that creates a branch between the plug 64 and the power supply 62, such that a portion of the AC power received through the plug 64 is carried to the power supply 62 and another portion of that AC power is carried to one or more components within the sterilization cabinet 2 that require AC power.

A controller 66 may be located in the control chamber 7. The controller 66 may receive DC power from the power supply 62. According to other embodiments, the controller 66 may utilize AC power. The controller 66 may provide a data connection to a wired and/or wireless network outside the sterilization cabinet 2. A cable 67 capable of carrying data transmissions to and from the controller 66 may extend outward from the controller 66, and a standard data connector may be connected to the end of the cable 67. The cable 67 extends out of the control chamber 7. The data connector 68 is configured to plug into a wall connector for a standard wired data network, such as but not limited to an Ethernet network. Instead of, or in addition to, the cable 67, the controller 66 may be connected to an antenna 70 configured to connect to one or more wireless data networks outside the sterilization cabinet 2. One such wireless data network may be, for example, a wireless data network following the WI-FI® protocol of the Wi-Fi Alliance of Austin, Texas, or a wireless data network following the BLUETOOTH® protocol of the Bluetooth SIG of Kirkland, Washington. One such wireless data network may be a cellular data network.

At least one UV sensor 80 may be positioned within the sterilization chamber 4. The UV sensor 80 may be placed vertically between the lower UV light source 50 and the upper UV light source 54. According to some embodiments, the UV sensor 80 may be positioned above the rotary table 20. According to other embodiments, the UV sensor 80 may be positioned below the rotary table 20. The UV sensor 80 is spaced laterally apart from the UV light sources 50, 54. According to some embodiments, the UV sensor 80 may be placed near an edge of the rotary table 20, at the rear of the sterilization chamber 4. However, the UV sensor 80 may be placed in any other suitable location within the sterilization chamber 4. The UV sensor 80 measures one or more characteristics of the UV light emitted from the UV light sources 50, 54 during the sterilization process, such as but not limited to power, wavelength, spectral energy distribution, and/or pulse time. Such measurements are transmitted from the UV sensor 80 to the controller 66, which may store those measurements and/or transmit those measurements to an external device. The measurement of one or more characteristics of the UV light emitted from the UV light sources 50, 54 may be used to verify that the application of pulsed UV light to an item to be sterilized met the requirements for sterilization, and that measurement may form part of the Batch Record, Lot History Record, and/or Device History Record for that sterilized item. The UV sensor 80 may receive DC power from the power supply 62, or may receive AC power from the power supply 62 or from a branch from the wire or wires 65, depending on the power needs of the particular UV sensor 80 utilized.

At least one airborne particle counter 90 may be positioned at any suitable location within the sterilization chamber 4. The airborne particle counter 90 measures the number, size, and/or other characteristics of airborne particles present in the sterilization chamber 4 during the sterilization process. Such measurements are transmitted from the airborne particle counter 90 to the controller 66, which may store those measurements and/or transmit those measurements to an external device. Measurements from the airborne particle counter 90 may be used to verify that the number, size or other characteristics of airborne particles present in the sterilization chamber 4 during the sterilization process did not exceed the number, size or other characteristics allowable by the requirements for sterilization, and those measurements may form part of the Batch Record, Lot History Record, and/or Device History Record for that sterilized item. The airborne particle counter 90 may receive DC power from the power supply 62, or may receive AC power from the power supply 62 or from a branch from the wire or wires 65, depending on the power needs of the particular airborne particle counter 90 utilized.

At least one viable microbe detector 100 may be positioned at any suitable location within the sterilization chamber 4. According to some embodiments, the viable microbe detector 100 may be part of the airborne particle counter 90. According to other embodiments, the viable microbe detector 100 may be a device that is separate from the airborne particle counter 90. The viable microbe detector 100 measures the viability, number, size, and/or other characteristics of microbes present in the sterilization chamber 4 during the sterilization process. Such measurements are transmitted from the viable microbe detector 100 to the controller 66, which may store those measurements and/or transmit those measurements to an external device. Measurements from the viable microbe detector 100 may be used to verify that the viability, number, size, and/or other characteristics of microbes present in the sterilization chamber 4 during the sterilization process did not exceed the viability, number, size, and/or other characteristics allowable by the requirements for sterilization, and those measurements may form part of the Batch Record, Lot History Record, and/or Device History Record for that sterilized item. The viable microbe detector 100 may receive DC power from the power supply 62, or may receive AC power from the power supply 62 or from a branch from the wire or wires 65, depending on the power needs of the viable microbe detector 100 utilized.

At least one temperature sensor 110 may be positioned at any suitable location within the sterilization chamber 4. The temperature sensor 110 measures the temperature within the sterilization chamber 4 during the sterilization process. Such measurements are transmitted from the temperature sensor 110 to the controller 66, which may store those measurements and/or transmit those measurements to an external device. Measurements from the temperature sensor 110 may be used to verify that the temperature within the sterilization chamber 4 during the sterilization process did not exceed the maximum temperature allowable by the requirements for sterilization, and those measurements may form part of the Batch Record, Lot History Record, and/or Device History Record for that sterilized item. The temperature within the sterilization chamber 4 during the sterilization process may be particularly important when sterilizing a container that has been filled with medication, as certain medications may have a much lower maximum temperature threshold than others. The temperature sensor 110 may receive DC power from the power supply 62, or may receive AC power from the power supply 62 or from a branch from the wire or wires 65, depending on the power needs of the temperature sensor 110 utilized.

At least one camera 120 may be positioned at any suitable location within the sterilization chamber 4. The camera 120 views and generates photographs and/or video of at least part of the interior of the sterilization chamber 4 during the sterilization process. Such photographs and/or video are transmitted from the camera 120 to the controller 66, which may store those photographs and/or video and/or transmit those photographs and/or video to an external device. According to some embodiments, the camera 120 may be fixed in place. According to other embodiments, the camera 120 may be movable about at least one axis, such as to tilt and/or pan. In such embodiments, the controller 66 is configured to send a command or commands to the camera 120 or a mount therefor to cause the camera 120 to rotate about at least one axis. Photographs and/or video from the camera 120 may be used to verify that the sterilization process is occurring normally, to troubleshoot issues within the sterilization chamber during sterilization, and for any other suitable purposes. The photographs and/or video generated by the camera 120 may form part of the Batch Record, Lot History Record, and/or Device History Record for that sterilized item. The camera 120 may receive DC power from the power supply 62, or may receive AC power from the power supply 62 or from a branch from the wire or wires 65, depending on the power needs of the camera 120 utilized.

At least one nebulizer or atomizer 130 optionally may be positioned at any suitable location within the sterilization chamber 4. The nebulizer or atomizer 130 introduces liquid droplets into the air within the sterilization chamber 4, and may act to minimize the size of those liquid droplets. According to some embodiments, the liquid droplets introduced into the air within the sterilization chamber 4 may be a sterilant, providing additional sterilization to items within the sterilization chamber 4 and/or reducing the number of living airborne microbes present within the sterilization chamber 4. According to other embodiments, the liquid droplets may be a substance or substances that enhance sterilization by changing a material's surface characteristics, such as but not limited to reflectivity, surface roughness, and organism clumping. According to other embodiments, the nebulizer or atomizer 130 may introduce liquid droplets into the air within the sterilization chamber 4, where the liquid is a mixture of two or more separate substances, and at least some of the liquid droplets are a mixture of two or more separate substances.

According to some embodiments, data connection between the controller 66 and at least one UV sensor 80, airborne particle counter 90, viable microbe detector 100, temperature sensor 110, camera 120, and/or nebulizer 130 may be a wired connection, such as an Ethernet connection. According to some embodiments, data connection between the controller 66 and at least one UV sensor 80, airborne particle counter 90, viable microbe detector 100, temperature sensor 110, camera 120, and/or nebulizer 130 may be a wireless connection, such as a connection using the WI-FI® protocol of the Wi-Fi Alliance of Austin, Texas, or a connection using the BLUETOOTH® protocol of the Bluetooth SIG of Kirkland, Washington.

The UV light sources 50, 54 may receive DC power from the power supply 62, or may receive AC power from the power supply 62 or from a branch from the wire or wires 65, depending on the power needs of the UV light sources 50, 54. Similarly, the motor that rotates the rotary table 20 may receive DC power from the power supply 62, or may receive AC power from the power supply 62 or from a branch from the wire or wires 65, depending on the power needs of the motor.

### Operation-Sterilization Chamber

Referring also to FIG. 9, a method 140 of operating the sterilization cabinet 2 is shown. First, at box 142, one or more PCCSs 42 are loaded into the sterilization chamber 4 of the sterilization cabinet 2. Such loading may be accomplished in any suitable manner. According to some embodiments, an operator opens the door 8, and places one or more PCCSs 42 onto the rotary table 20. According to other embodiments, some of which are described below, one or more mechanisms are utilized to load one or more PCCSs 42 onto the rotary table 20. The PCCSs 42 may be loaded into the sterilization chamber 4 by themselves, according to some embodiments. According to other embodiments, one or more PCCSs 42 are placed within a nest 30 before being loaded into the sterilization chamber 4. In this document, the description of handling a PCCS 42 includes the handling of the combination of a PCCS 42 and associated nest 30, where a nest 30 is useful to hold the PCCS 42 in place on the rotary table 20 or other carrier.

Next, at box 144, the one or more PCCSs 42 that were loaded into the sterilization chamber 4 in box 142 are moved into sterilization position. That sterilization position may be located under, above, or between one or more UV light sources 50, 54, if the one or more PCCSs 42 were loaded into the sterilization chamber 4 at a different position. According to some embodiments, one or more PCCSs 42 are placed onto the rotary table 20 in box 142, and the rotary table 20 is rotated in box 142 to place the one or more PCCSs 42 in sterilization position.

Optionally, at box 146, a positive confirmation is made that the one or more PCCSs 42 have been placed in sterilization position. According to some embodiments, such a confirmation may be made by an operator using the at least one camera 120 to view the one or more PCCSs 42 within the sterilization chamber 4. According to other embodiments, such a confirmation may be made automatically without operator intervention, such as by image analysis, and/or the use of a mechanical or electrical marker on the rotary table 20 that is associated with the position of the one or more PCCSs 42, and a sensor in the sterilization chamber 4 that determines whether that mechanical or electrical marker is in a location associated with the sterilization position.

Next, at box 148, at least one UV light source 50, 54 is activated to generate UV light as described above, and to illuminate the one or more PCCSs 42 in the sterilization position. According to some embodiments, pulsed UV light is generated at box 148. After completion of box 148, the PCCSs 42 in the sterilization position are sterile. A container such as a PCCS 42 is “sterile” when the probability is less than one out of one million that it is contaminated with replicating microorganisms, according to the World Health Organization. That definition of the term “sterile” is used in this document.

At box 150, one or more measurements of conditions inside the sterilization chamber 4 may be made before, during and/or after the performance of box 148. As described above, at least one UV sensor 80 may measure UV light that was produced by the at least one light source 50, 54 during box 148. At least one airborne particle counter 90 may measure airborne particles within the sterilization chamber 4 before, during and/or after box 148. At least one viable microbe detector 100 may measure viable microbes within the sterilization chamber 4 before, during and/or after box 148. At least one temperature sensor 110 may measure the temperature within the sterilization chamber 4 before, during and/or after box 148. At least one camera 120 may visually monitor the interior of the sterilization chamber 4 before, during and/or after box 148. The measurement or measurements taken in box 150 may be transmitted to the controller 66, and from there to an external computer, network or system.

Next, at box 152, the rotary table 20 is rotated to an unloading position. According to some embodiments, the rotary table 20 rotates in one direction, such that the rotary table 20 when in unloading position is in a different rotational position from its position in box 142 in which the PCCSs 42 were loaded into the sterilization chamber 4. According to other embodiments, the rotary table 20 rotates back to its initial position in box 142; in such embodiments, the unloading position of the rotary table 20 is substantially the same as the loading position of the rotary table 20 in box 142.

Next, at box 154, the PCCSs 42 are removed from the sterilization chamber 4. As described in greater detail below, the sterilized PCCSs 42 must be handled in a manner that preserves their sterility. Such removal of the PCCSs 42 in box 154 is performed accordingly.

Optionally, at box 156, the measurements of box 150 are compared to the specifications associated with those measurements. If the measurements of box 150 fall within specifications, the PCCSs 42 sterilized at box 148 are accepted in box 158. If one or more of the measurements of box 150 do not fall within specifications, the PCCSs 42 sterilized at box 148 are rejected in box 160, because the measurements of box 150 did not verify that the PCCSs 42 were in fact sterilized.

### Operation—Workflow

Referring also to FIG. 10,, the sterilization cabinet 2 may be located in a cleanroom 170. In the United States, the requirements for a clean room are set forth in ISO 14644-1. That standard sets forth 9 different classes of clean room, ranging from ISO Class 1 to ISO Class 9. ISO 14644-1 defines each class in terms of the maximum allowable concentrations of particles having defined diameters. The largest concentrations of particulates are allowable in an ISO Class 9 clean room; the smallest concentrations of particulates are allowable in an ISO Class 1 clean room. In Europe, the requirements for a clean room used for drug and medical device manufacturing are set forth in the “EudraLex—Volume 4—Good Manufacturing Practice (GMP) Guidelines.” That EU GMP sets forth four grades of clean room, ranging from Grade A to Grade D. Grades A and B are approximately equivalent to ISO Class 5. Grade C is approximately equivalent to ISO Class 7. Grade D is approximately equivalent to ISO Class 8. An ISO Class 5/EU GMP Grade A space may be referred to as a “critical area,” where sterilized drug product, as well as any containers and closures, are exposed to environmental conditions that must be designed to maintain product sterility. Activities conducted in such areas include manipulations (e.g., aseptic connections, sterile ingredient additions) of sterile materials prior to and during filling and closing operations. According to other embodiments, the sterilization cabinet 2 may be located in a space other than a cleanroom 170, such as a hospital operating room.

Different levels of ISO-defined space may be present in one cleanroom. An advantage of the present invention is that it allows for PCCSs 42 to be loaded into the sterilization chamber 4 from a less-stringent ISO class cleanroom, which is less expensive and simpler to manage, and then be sterilized to an ISO Class 5 or stricter standard prior to further processing.

Referring also to FIG. 11, a schematic view of an environment 200 in which the sterilization cabinet 2 is used is shown. The sterilization cabinet 2 may be placed inside a first cleanroom 202, which itself may be located in or adjacent to a second cleanroom 204. The first cleanroom 202 may be maintained at a cleaner ISO class than the second cleanroom 204. According to some embodiments, the first cleanroom 202 is an ISO Class 7 cleanroom, and the second cleanroom 204 is an ISO class 8 cleanroom. The sterilization chamber 2 may constitute an ISO Class 6 chamber in and of itself. According to other embodiments, the sterilization cabinet 2 is placed in a third cleanroom or clean volume that is located within, or adjacent to, the first cleanroom 202. In such embodiments, the third cleanroom is maintained at a cleaner ISO class than the first cleanroom 202. For example, where the first cleanroom is an ISO Class 7 cleanroom, the third cleanroom may be an ISO Class 6 cleanroom.

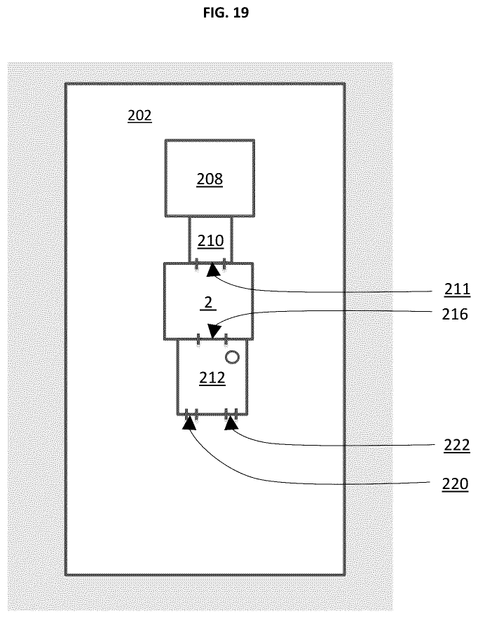

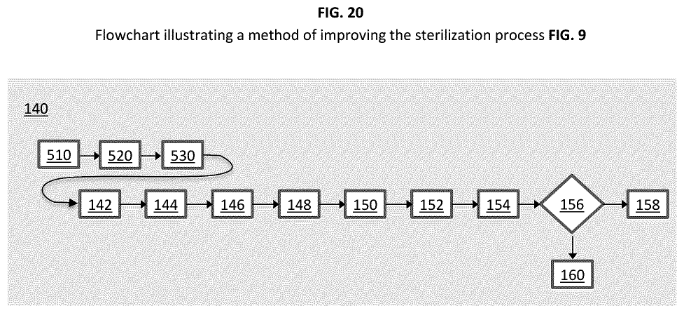

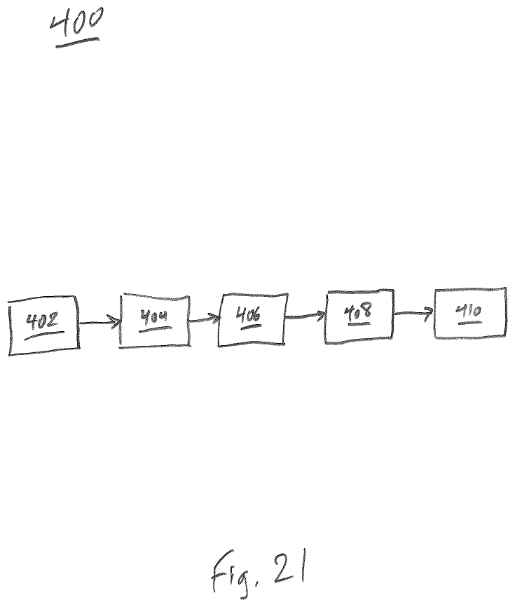

Referring also to FIG. 19, a holding area 208 in the first cleanroom 202 holds PCCSs 42 to be sterilized. A transport mechanism 210 may be provided to move PCCSs 42 from the holding area 208 to the sterilization cabinet 2. According to such embodiments, an entrance shutter 211 may be located at the entrance to the sterilization chamber 4. Each shutter used in conjunction with the sterilization process substantially prevents airborne microbes, particulates, and other matter from moving from a less-clean ISO Class space into a more-clean ISO Class space. Pressure differentials, such as a higher pressure in a more-clean space than an adjoining less-clean space, air curtains, and/or other techniques may assist in substantially preventing airborne microbes, particulates, and other matter from moving into the more-clean space. When a shutter is closed, that shutter substantially seals the more-clean space from the less-clean space. When the shutter is open, pressure differentials, air curtains, and/or other techniques substantially prevent unwanted airborne matter from moving into the more-clean space. The entrance shutter 211 is open long enough to allow the transport mechanism 210 to place PCCSs 42 into the sterilization chamber 4 at a loading position, then it is closed. According to other embodiments, one or more operators manually place PCCSs 42 to be sterilized into the sterilization cabinet 2 through the entrance shutter 211, such that a transport mechanism 210 is not provided. According to other embodiments, one or more operators manually place PCCSs 42 to be sterilized into the sterilization cabinet 2 through the door 8. Such embodiments may be useful where the number of PCCSs 42 to be sterilized is relatively low.

A fill-and-finish module 212 may be connected to the sterilization chamber 4. The fill-and-finish module 212 may be maintained at a cleaner ISO class than the sterilization chamber 4. For example, where the sterilization chamber 4 is a clean volume or is located in a cleanroom maintained at ISO Class 6, the fill-and-finish module 212 may be maintained at ISO Class 5. The fill-and-finish module 212 may constitute an ISO Class 5 chamber in and of itself. According to other embodiments, the fill-and-finish module 212 is placed in a fourth cleanroom or clean volume that is located within, or adjacent to, the first cleanroom 202 or the third cleanroom 206 (if used). In such embodiments, the fourth cleanroom is maintained at a cleaner ISO class than the first cleanroom 202 and the third cleanroom 206 (if used). A fill-and-finish module entrance shutter 216 may be located between the sterilization chamber 4 and the fill-and-finish module 212. The fill-and-finish module entrance shutter 216 may be closed until the PCCSs 42 located in the sterilization chamber 4 have been sterilized.

After the PCCSs 42 have been sterilized in the sterilization chamber 4, the fill-and-finish module entrance shutter 216 is opened, and the PCCSs 42 are transferred to the fill-and-finish module 212, after which the fill-and-finish module entrance shutter 216 is closed. Such transfer may be accomplished by standard means, such as but not limited to a movable pusher bar; a stationary pusher bar against which the PCCSs 42 are urged during rotation of the rotary table 20, causing the PCCSs 42 to move radially outward into the fill-and-finish module; and a robotic arm. The fill-and-finish module 212 may perform one or more functions. As used in this document, the term “medication” means at least one of liquid medication, powder medication, solid medication, pills, gel, aerosol, emulsion, syrup, cream, lotion, or ointment. According to some embodiments, the fill-and-finish module 212 fills the sterilized PCCSs 42 received from the sterilization chamber 4 with medication. The use of the fill-and-finish module 212 in conjunction with the sterilization chamber 4 is particularly useful when the medication to be placed into one or more PCCSs 42 is easily degradable by exposure to heat, radiation, ozone, ethylene oxide, or UV light. Because the PCCSs 42 are sterilized immediately before being moved into the fill-and-finish module 212, and the fill-and-finish module 212 is maintained in an ISO Class 5 space that is suitable for the preparation of medication, the fill-and-finish module 212 is able to fill the sterile PCCSs 42 with medication, maintain sterility, and preserve the medication from the adverse effects that it may experience from exposure to UV light or other sterilization methods. Biologics are particularly vulnerable to damage during conventional sterilization processes. To put it another way, medication that is known to be sterile is placed into a sterile PCCS 42, without the need to expose the medication to a sterilization process; as a result, the medication cannot be damaged by the sterilization process.

According to some embodiments, the fill-and-finish module 212 finishes the PCCSs 42 after they are filled. “Finishing” refers to the process of closing each PCCS 42 such that it maintains the sterility of the medication that it contains. Finishing can refer to sealing a vial or single-use ampule that holds medication, capping a syringe that is filled with medication, sealing a blister cavity that holds medication, scaling strip packaging that holds a plurality of pills, capping and scaling a bottle that holds liquid; or sealing a sachet bag or IV bag that holds medication.

According to some embodiments, the fill-and-finish module 212 inspects the PCCSs 42 after they have been finished. Such inspecting may include checking for appropriate fill of a vial, syringe, sachet bag or IV bag, ensuring that each strip package includes the correct number of pills, and/or ensuring that a blister cavity contains the correct amount of medication or number of pills. The inspection also may include an evaluation of the information gathered at box 150 regarding conditions in the sterilization chamber 4 during sterilization, particularly if it takes time to process that information. If that information indicates that conditions in the sterilization chamber 4 did not meet specifications, the PCCSs 42 associated with that information fail inspection. According to other embodiments, the evaluation of the information gathered at box 150 may occur prior to the movement of the PCCSs 42 associated with that information into the fill-and-finish module 212. In such embodiments, those PCCSs 42 fail verification, and are moved out of the sterilization chamber 4 without entering the fill-and-finish module 212. Alternately, in such embodiments, the failure of the sterilization chamber 4 to meet requirements for sterilization of one or more PCCSs 42 causes the sterilization chamber 4 and/or the fill-and-finish module 212 to cease operation until an operator can determine the cause of such failure and correct it.

After inspection, the fill-and-finish module 212 moves the filled PCCSs 42 out of the fill-and finish module 212, back into the first cleanroom 202. According to some embodiments, filled PCCSs 42 that pass inspection may exit the fill-and-finish module 212 through a pass shutter 220. Referring also to FIG. 19, filled PCCSs 42 that fail inspection may exit the fill-and-finish module 212 through a fail shutter 222. In this way, PCCSs 42 that have passed inspection are clearly separated from PCCSs 42 that have failed inspection. According to other embodiments, separate shutters 220, 222 for PCCSs 42 that passed and that failed inspection are not used, and all filled PCCSs 42 pass through one single exit shutter. The fill-and-finish module 212 indicates to workers or to a later part of the process whether individual PCCSs 42 that exit the single exit shutter have passed or failed inspection.

The fill-and finish module 212 may itself include a rotary table 224 that moves PCCSs 42 from filling to finishing to inspection. Optionally, the rotary table 224 may have a larger diameter than the rotary table 20 in the sterilization chamber 4. According to other embodiments, the fill-and finish module 212 includes a conveyor belt or similar item, instead of or in addition to a rotary table 224, to move PCCSs 42 within the fill-and finish module 212.

It will be appreciated that the sterilization cabinet 2 may include one or more additional doors 8 or other structures and mechanisms to separate the sterilization chamber 4 from the space outside, in order to facilitate sterilization of PCCSs 42 and their later processing as described in greater detail below. According to some embodiments, the door 8 is as described above, and a shutter is movable relative to an aperture in the wall of the sterilization chamber 4. In such embodiments, PCCSs 42 to be sterilized are placed into the sterilization chamber 4 by opening the door 8, and the door is closed after loading. After sterilization, the shutter 216 is automatically or manually moved to an open position to allow PCCSs 42 to be moved into the fill-and-finish module 212, after which the shutter is automatically or manually moved to a closed position. According to other embodiments, the door 8 of the sterilization chamber 4 instead may be a shutter, which is movable relative to an aperture in the wall of the sterilization chamber 4. The shutter is opened for loading PCCSs 42 to be sterilized into the sterilization chamber, and is closed after loading. The shutter may be automatically or manually moved to an open position to allow PCCSs 42 to be loaded into the sterilization chamber 4, after which the shutter is automatically or manually moved to a closed position. The use of a shutter instead of a door 8 may facilitate automatic and/or mechanical loading of PCCSs 42 into the sterilization chamber, such as by the use of a transport mechanism 210.

Turning to the operation of the system described above, referring also to FIG. 12, that method 230 begins in box 232 with the transport of PCCSs 42 to be sterilized from the holding area 208 to the sterilization chamber 4. As described above, such transport may be performed manually or may be performed by a system that is at least partially automated. Next, in box 234, the door 8 or shutter 218 through which PCCSs 42 were loaded into the sterilization chamber 4 is closed. Next, at box 236, the method 140 described above for sterilization of PCCSs 42 in the sterilization chamber 4 is performed.

Next, at box 238, the shutter 216 between the sterilization chamber 4 and the fill-and-finish module 212 is opened. At box 240, the sterilized PCCSs 42 are transferred from the sterilization chamber 4 into the fill-and-finish module 212. Where the fill-and-finish module 212 includes a rotary table 224, the sterilized PCCSs 42 are transferred onto that rotary table 224.

At box 242, the fill-and-finish module 212 fills the sterilized PCCSs 42 received from the sterilization chamber 4 with medication, as described above. At box 244, the fill-and-finish module 212 finishes the filled PCCSs 42, as described above. At box 246, the fill-and-finish module 212 inspects the finished PCCSs 42. After inspection, the finished PCCSs 42 exit the fill-and-finish module 212 at box 248. The exiting of the finished PCCSs 42 at box 248 includes the opening of at least one shutter 220 to allow the exit of the finished PCCSs 42. Optionally, at box 248, PCCSs 42 that have passed inspection exit the fill-and-finish module 212 through a first shutter 220, and PCCSs 42 that have failed inspection exit the fill-and-finish module 212 through a second shutter 222. A shutter 220, 222 separates a decontaminated unit supplied with ISO Class 5 or higher air quality that provides uncompromised, continuous isolation of its interior from the external environment. At box 250, either or both shutter 220, 222 that opened at box 248 are closed.

### Linear Carrier

Referring also to FIG. 13, one or both of the rotary table 20 in the sterilization chamber 4 and the rotary table 224 in the fill-and-finish module 212 is omitted, and in its place a linear carrier may be used. A first linear carrier 252 may be utilized in the sterilization chamber 4, and/or a second linear carrier 254 may be utilized in the fill-and-finish module 212. The linear carriers 252, 254 may be belt conveyors, roller conveyors, wire-or cable-driven containers, or any other suitable type of carrier. Advantageously, the linear carrier or carriers 252, 254, may be transparent, may be at partially a mesh, or include a plurality of apertures to allow UV light from the UV light sources 50, 54 to expose the PCCSs 42 carried in the linear carrier or carriers 50, 54 to UV light from the top and bottom. The use of the linear carrier or carriers 252, 254 may facilitate a higher-volume process for sterilization, filling, finishing and inspecting PCCSs 42.

Where the rotary table 20 in the sterilization chamber 4 is replaced with a first linear carrier 252, the sterilization cabinet 2 and the method 140 of operating the sterilization cabinet 2 as set forth in FIG. 9, the environment 200 of FIG. 11 and the description thereof is substantially the same, and the method 230 of FIG. 12 and the description thereof is substantially the same, except as set forth in this paragraph. At box 144 of FIG. 9, the one or more PCCSs 42 that were loaded into the sterilization chamber 4 in box 142 are moved into sterilization position by actuating the first linear carrier 252 to place the one or more PCCSs 42 in sterilization position. The first linear carrier 252 may do so by moving continuously, or may move intermittently (either automatically or under manual control) to carry the one or more PCCSs 42 from an initial position to the sterilization position. At box 146, a mechanical or electrical marker on the first linear carrier 252 may be associated with the position of the one or more PCCSs 42. At box 152, the first linear carrier 252 is actuated to move the sterilized PCCSs 42 to an unloading position. The first linear carrier 252 may do so by moving continuously, or may move intermittently (either automatically or under manual control) to carry the one or more PCCSs 42 from the sterilization position to an unloading position. According to some embodiments, the first linear carrier 252 moves in one direction, such that the unloading is in a different linear position from the position in box 142 where the PCCSs 42 were loaded onto the first linear carrier 252. According to other embodiments, the first linear carrier 252 slides or moves back to its initial position in box 142; in such embodiments, the unloading position of the first linear carrier 252 is substantially the same as the loading position of the first linear carrier 252 in box 142. Where the first linear carrier 252 and the second linear carrier 254 are both utilized, transfer of the PCCSs 42 to the fill-and-finish module 212 from the sterilization chamber 4 is straightforward, and the movable pusher bar; stationary pusher bar against which the PCCSs 42 are urged during rotation of the rotary table 20, and robotic arm described above are not needed. There is no need to move sterile PCCSs 42 outward to an edge of the rotary table 20; rather, sterile PCCSs 42 travel out of the sterilization chamber 4 on the first linear carrier 252 and are smoothly picked up by the second linear carrier 254. According to some embodiments, a single linear carrier 252 extends between the sterilization chamber 4 and the fill-and-finish module 212 for even smoother material handling. Where a rotary table 20 is used in the sterilization chamber 4 and a second linear carrier 254 is used in the fill-and-finish module 212, the movable pusher bar; stationary pusher bar against which the PCCSs 42 are urged during rotation of the rotary table 20, or robotic arm described above may be used to urge the sterile PCCSs 42 radially off the rotary table 20 onto the second linear carrier 254.

Referring also to FIG. 16, one or more UV light reflectors 302, filters 304, lenses 306, opaque barriers 308, and/or other optical elements such as mirrors and/or diffraction gratings may be used to manage UV light inside the sterilization chamber 4 during sterilization. As used in this document, managing UV light, management of UV light, and similar phrases mean the control of characteristics of UV light and the locations where that UV light is directed in the sterilization chamber 4. Such optical elements may be utilized regardless of whether a rotary table 20 or one or more linear carriers 252, 254 are used to transport PCCSs 42 within the sterilization chamber 4. Management of UV light characteristics and the locations where UV light is directed in the sterilization chamber 4 by the use of such optical elements may be more helpful where one or more linear carriers 252, 254 are utilized, and where at least a part of each linear carrier 252, 254 is not substantially transparent to UV light.

### Entry and/or Exit Tunnels

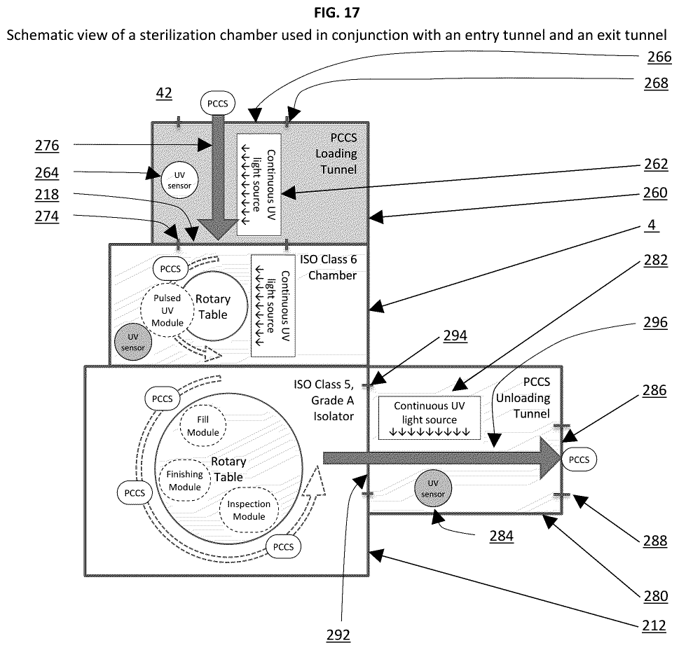

Referring also to FIG. 17, an entry tunnel 260 may be utilized for loading of PCCSs 42 into the sterilization chamber 4. The entry tunnel 260 pre-sanitizes empty PCCSs 42 prior to their sterilization in the sterilization chamber 4, thereby reducing bioburden from the processing environment even further. The entry tunnel 260 includes at least one entry tunnel UV light source 262 therein that is on, and emitting light, at least during some or all of the times when empty PCCSs 42 are positioned in the entry tunnel 260. According to some embodiments, at least one entry tunnel UV light source 262 may be one or more LEDs emitting UV light at a peak wavelength of substantially 275-280 nm. Other sources of continuous UV light may be utilized. The at least one entry tunnel UV light source 262 does not and need not sanitize the empty PCCSs 42 completely; rather, the at least one entry tunnel UV light source 262 is useful for killing surface bacteria and other bioburden associated with the empty PCCSs 42, to allow the sterilization chamber 4 to be even more effective at ensuring sterilization of the PCCSs 42. Optionally, at least one entry tunnel UV sensor 264 may be positioned in the entry tunnel 260 to verify that the at least one entry tunnel UV light source or sources 262 has illuminated the empty PCCSs 42 to the degree expected. According to some embodiments, a transport mechanism 210 is located within the entry tunnel 260. According to other embodiments, the transport mechanism is configured to transport empty PCCSs 42 to the entrance of the entry tunnel 260.

An entry tunnel shutter 266 may be located at an entrance opening 268 of the entry tunnel 260. When closed, the entry tunnel shutter 266 blocks substantially all UV light from escaping through the entrance opening 268. This blockage is important to the safety and health of workers who may be present in the vicinity of the entrance opening 268 during operation of the entry tunnel 260. The at least one entry tunnel UV light source 262 may be interlinked with the entry tunnel shutter 266 to ensure that the at least one entry tunnel UV light source 262 is off when the entry tunnel shutter 266 is open. As one example, according to some embodiments, an entrance tunnel switch is actuated when the entry tunnel shutter 266 is in the open position, and actuation of that entrance tunnel switch causes each entry tunnel UV light source 262 in the entry tunnel 260 to turn off. According to other embodiments, the entry tunnel shutter 266 is not used, and baffles, curtains, and/or other forms of light reduction may be placed in the entry tunnel 260 to restrict UV light from exiting the entrance opening 268. Such forms of light reduction may be useful in high-volume and/or automated processes in which workers are not in frequent proximity to the entrance opening 268 of the entry tunnel 260.

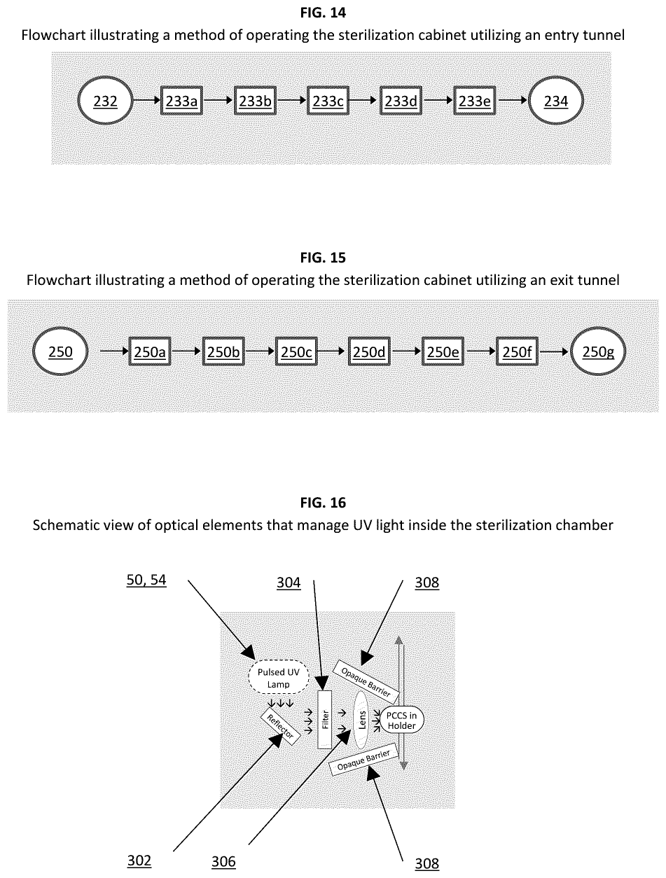

A entrance shutter 218 is located at the exit 274 of the entry tunnel 260, which connects to the sterilization chamber 4. An entry tunnel linear carrier 276, such as a conveyor belt, is used to convey empty PCCSs 42 from the entrance 268 of the entry tunnel 260 to the entrance of the sterilization chamber 4. The entry tunnel linear carrier 276 may run generally continuously, or may be actuated to move empty PCCSs 42 through the entry tunnel 260 and into the sterilization chamber 4 intermittently. According to some embodiments, the entrance shutter 218 is omitted. In such embodiments, the entry tunnel linear carrier 276 may carry empty PCCSs 42 into the sterilization chamber 4 continuously, and that entry tunnel linear carrier 276 may be substantially the same as the linear carrier used in other embodiments described herein. Embodiments in which the entrance shutter 218 is omitted may be particularly useful for high volume sterilization of PCCSs 42.

Where the entry tunnel 260 is used, the sterilization cabinet 2 and the method 140 of operating the sterilization cabinet 2 as set forth in FIG. 9, the environment 200 of FIG. 11 and the description thereof is substantially the same, and the method 230 of FIG. 12 and the description thereof is substantially the same, except as set forth in this paragraph and in FIG. 14. At box 232 of FIG. 12, empty PCCSs 42 to be sterilized are transported from the holding area 208 to the entrance opening 268 of the entry tunnel 260. Next, at box 233a, the entry tunnel shutter 266 is closed, and at least one entry tunnel UV light source 262 is activated. The empty PCCSs 42 in the entry tunnel 260 are exposed to UV light from the at least one entry tunnel UV light source 262. At box 233b, the empty PCCSs 42 are moved through the entry tunnel 260 toward the sterilization chamber 4, such as by the entry tunnel linear carrier 276. At box 233c, the entry tunnel UV sensor 264 senses one or more characteristics of the UV light emitted by the at least one entry tunnel UV light source 262, and transmits those characteristics to the controller 66 or other device. Measurements from the entry tunnel UV sensor 264 are used to verify the amount of UV light to which the empty PCCSs 42 were exposed, and may form part of the Batch Record, Lot History Record, and/or Device History Record for those PCCSs 42. At box 233d, the shutter 218 between the entry tunnel 260 and the sterilization chamber 4 is opened. At box 233c, at least one of the empty PCCSs 42 in the entry tunnel 260 is loaded into the sterilization chamber 4 from the entry tunnel 260. As in FIG. 12, in box 234, the shutter 218 through which PCCSs 42 were loaded into the sterilization chamber 4 is closed. The remainder of the method 230 of FIG. 12 proceeds as described above.

Referring also to FIG. 17, an exit tunnel 280 may be utilized for unloading PCCSs 42 from the fill-and-finish chamber 212. The exit tunnel 280 maintains sanitation of filled and finished PCCSs 42 after their sterilization in the sterilization chamber 4, thereby preventing ingress of contamination into the processing environment and the fill-and-finish chamber 212. The exit tunnel 280 includes at least one exit tunnel UV light source 282 therein that is on, and emitting light, at least during some or all of the times when filled and finished PCCSs 42 are positioned in the exit tunnel 280. According to some embodiments, at least one exit tunnel UV light source 282 may be one or more LEDs emitting UV light at a peak wavelength of substantially 275-280 nm. Other sources of continuous UV light may be utilized. The at least one exit tunnel UV light source 282 does not and need not sanitize the filled and finished PCCSs 42 completely; rather, the at least one exit tunnel UV light source 282 is useful for killing surface bacteria and other bioburden that may enter the exit tunnel from the less-clean first cleanroom 202. The peak wavelength of the UV light emitted from the at least one exit tunnel UV light source 282, the lumen intensity of that UV light, and other properties of that UV light are selected to ensure that the UV light from the at least one exit tunnel UV light source 282 does not degrade or damage the medication held within the filled and finished PCCSs 42. Optionally, at least one exit tunnel UV sensor 284 may be positioned in the exit tunnel 280 to verify that the at least one exit tunnel UV light source 282 has illuminated the empty PCCSs 42 to the degree expected.

An exit tunnel shutter 286 may be located at an exit opening 288 of the exit tunnel 280. When closed, the exit tunnel shutter 286 blocks substantially all UV light from escaping through the exit opening 288. This blockage is important to the safety and health of workers who may be present in the vicinity of the exit opening 288 during operation of the exit tunnel 280. The at least one exit tunnel UV light source 282 may be interlinked with the exit tunnel shutter 286 to ensure that the at least one exit tunnel UV light source 282 is off when the exit tunnel shutter 286 is open. As one example, according to some embodiments, an exit tunnel switch is actuated when the exit tunnel shutter 286 is in the open position, and actuation of that switch causes each exit tunnel UV light source 282 in the exit tunnel 280 to turn off. According to other embodiments, the exit tunnel shutter 286 is not used, and baffles, curtains, and other forms of light reduction are placed in the exit tunnel 280 to restrict UV light from exiting the exit opening 288. Such forms of light reduction may be useful in high-volume and/or automated processes in which workers are not in frequent proximity to the exit opening 288 of the exit tunnel 280.

A second internal shutter 292 is located at the entrance 294 of the exit tunnel 280, which connects to the sterilization chamber 4. According to some embodiments, where the exit tunnel 280 is used, the exit from the sterilization chamber 4 is the second internal shutter 292. An exit tunnel linear carrier 296, such as a conveyor belt, is used to convey filled and finished PCCSs 42 from the entrance 294 of the exit tunnel 280 to the exit opening 288. The exit tunnel linear carrier 296 may run generally continuously, or may be actuated to move filled and finished PCCSs 42 out of the sterilization chamber 4 and through the exit tunnel 280 intermittently. According to some embodiments, the second internal shutter 292 is omitted. In such embodiments, the exit tunnel linear carrier 296 may carry filled and finished PCCSs 42 out of the sterilization chamber 4 generally continuously, and that exit tunnel linear carrier 296 may be substantially the same as linear carriers described with regard to other embodiments. Embodiments in which the second internal shutter 292 is omitted may be particularly useful for high volume sterilization of PCCSs 42.

Where the exit tunnel 280 is used, the sterilization cabinet 2 and the method 140 of operating the sterilization cabinet 2 as set forth in FIG. 9, the environment 200 of FIG. 11 and the description thereof is substantially the same, and the method 230 of FIG. 12 and the description thereof is substantially the same, except as set forth in this paragraph and in FIG. 15. After box 250, at box 250a, filled and finished PCCSs 42 move into the exit tunnel 280. At box 250b, the exit tunnel UV light source 282 is activated, if it was previously in the off state. The filled and finished PCCSs 42 in the exit tunnel 280 are exposed to UV light from the exit tunnel UV light source 282. At box 250c, the filled and finished PCCSs 42 are moved through the exit tunnel 280 away from the sterilization chamber 4, such as by the exit tunnel linear carrier 296. At box 250d, the exit tunnel UV sensor 284 senses one or more characteristics of the UV light emitted by the at least one exit tunnel UV light source 282, and transmits those characteristics to the controller 66 or other device. Measurements from the exit tunnel UV sensor 284 are used to verify one or more characteristics of UV light to which the empty PCCSs 42 were exposed by the exit tunnel UV light source 282, and may form part of the Batch Record, Lot History Record, and/or Device History Record for those PCCSs 42. At box 250e, the exit tunnel shutter 286 at the exit opening 288 is opened. At box 250f, at least one of the filled and finished PCCSs 42 in the exit tunnel 280 is removed from the exit tunnel 280. Such removal may be performed manually or automatically. At box 250g, the exit tunnel shutter 286 is then closed again. It should be noted that where the optional box 248 is performed, the exit tunnel 280 may be connected to the sterilization chamber via the first shutter 220, through which PCCSs 42 that have passed inspection exit. The second shutter 222 may empty onto a table, bin or other container to allow for further inspection of rejected finished PCCSs 42 for troubleshooting purposes, and/or for their disposal.

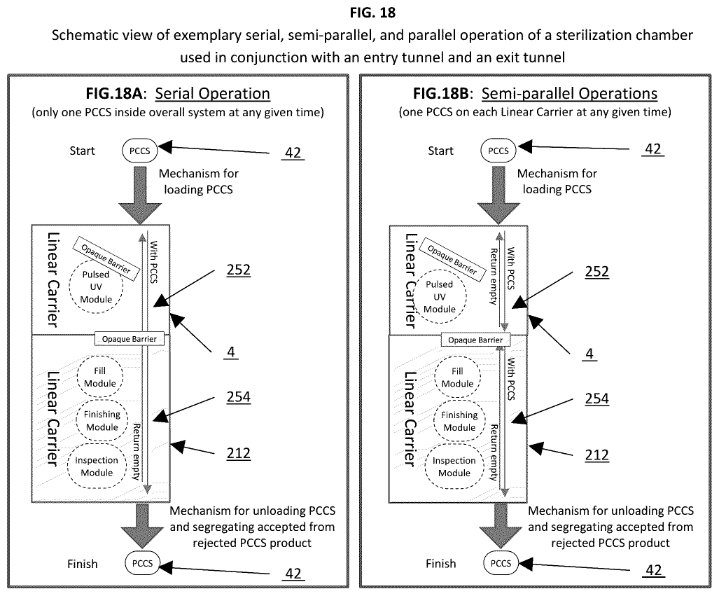

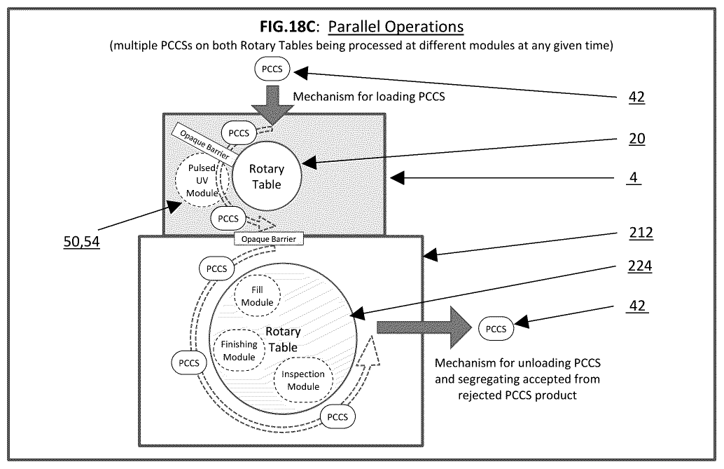

The systems and methods described above may be configured to process PCCSs 42 in a serial, semi-parallel, or parallel manner. Referring to FIG. 18A, serial operation of a sterilization chamber 4 with an entry tunnel 260 and an exit tunnel 280 is shown. As used in this document, “serial processing” or “serially processing” PCCSs 42 means that one and only one PCCS 42 is present at any given time within the combination of the sterilization chamber 4 and the fill-and-finish module 212. Referring to FIG. 18B, semi-parallel operation of a sterilization chamber 4 with an entry tunnel 260 and an exit tunnel 280 is shown. As used in this document, “semi-parallel processing” of PCCSs 42 means that one and only one PCCS 42 may be present in the sterilization chamber 4 at any given time, one and only one different PCCS 42 may be present in the fill-and-finish module 212 at any given time, and one PCCS 42 may be (but is not required to be) in the sterilization chamber 4 at the same time that a different PCCS 42 is present in the fill-and-finish module 212. Semi-parallel processing may be useful where a linear carrier 252 is used to move PCCSs 42 through the sterilization chamber 4, and a different linear carrier 254 is used to move PCCSs 42 through the fill-and-finish module 212. Referring to FIG. 18C, parallel operation of a sterilization chamber 4 with an entry tunnel 260 and an exit tunnel 280 is shown. As used in this document, “parallel processing” of PCCSs 42 means that multiple PCCSs 42 may be (but are not required to be) located in the sterilization chamber 4 at the same time, that multiple PCCSs 42 may be located in the fill-and-finish module 212 at the same time, and that one or more PCCSs 42 may be located in the sterilization chamber 4 at the same time that one or more PCCSs 42 are located in the fill-and-finish module 212.

Other products and items which may require sterilization, or a reduction of bioburden short of sterilization, include (a) aseptic processing and preservation enhancement equipment, fixtures, and supplies, including but not limited to: isolators, restricted access barriers (RABs), formulation and mixing equipment, fill and seal equipment, packaging equipment, and equipment and fixtures used to load and transfer products, tools and supplies; (b) medical devices, including but not limited to disposable devices and instruments, reusable devices and instruments, resposable devices and instruments, permanent and semi-permanent implants, machines and apparatuses, and their accessories; (c) sterile medical supplies, including but not limited to saline, bandages and wraps, swabs, sample containers, stoppers, syringes, kitted supplies and tools, markers, and gloves; (d) foods which require the elimination of microbes for preservation and prevention of spoilage; (e) beverages, and their containers, which require the elimination of microbes for preservation and prevention of spoilage; and (f) cosmetics which require the elimination of microbes for preservation and prevention of spoilage, including but not limited to liquids, gels, ointments, and powders. The sterile or decontaminated packaging of these products and items are also referred to as Primary Container Closure Systems (PCCSs). The system and method described above may be utilized to sterilize, or reduce bioburden short of sterilization, for such PCCSs. While the invention above has been described in terms of its use for sterilizing containers of parenteral medication, with or without the use of a nest or nests 30, the invention may be used for sterilizing medical devices, food products, cosmetics, and/or other products that have a need to be sterilized.

Prior to sterilization, empty PCCSs 42 may sit in a warehouse or even outdoors for a prolonged period of time. Purchasing PCCSs 42 in bulk from a vendor typically results in a lower unit price for each PCCS 42, where the larger the number purchased, the less the unit price is. For this reason, manufacturers may purchase enough empty PCCSs 42 at one time to last for weeks, months or even years of anticipated production. After delivery to the parenteral manufacturer, those PCCSs 42 are stored in packaging, in a cleaned and unsterilized condition, or in a cleaned and sterilized condition.

### Enhanced Sterilization with Pulsed UV Light and Hydrogen Peroxide

Pulsed UV light may be used to enhance sterilization of PCCSs 42 in a traditional hydrogen peroxide sterilization process, and hydrogen peroxide may be used to enhance sterilization of PCCSs using pulsed UV light as described above.

It is more desirable for PCCSs 42 to be stored in a cleaned and unsterilized condition; that way, multi-layered packaging is not required to protect and move the pre-sterilized PCCSs 42 into an aseptic processing environment without introducing contaminants. Furthermore, elaborate equipment and processes are not required to move the PCCSs into the aseptic processing environment without introducing contaminants. Using this more desirable approach, single-layer packaging can be used in a storage location such as a warehouse for a long period of time prior to being filled.

Referring also to FIG. 20, a method 500 is shown, which adds additional steps to the method 140 described above with reference to FIG. 9 in order to further improve the sterilization process. Prior to sterilization with the sterilization cabinet 2 and the associated method 140 described above, at box 510, the PCCSs 42 are filled with liquid hydrogen peroxide (H2O2). According to some embodiments, that filling may take place prior to the time at which the PCCSs 42 are placed in storage. In that way, the liquid H2O2 in the PCCSs 42 attacks any microorganisms that may enter, and do so days, weeks or even months before those PCCSs 42 are sterilized with the sterilization cabinet 4. According to other embodiments, the PCCSs 42 may be filled with liquid H2O2 prior to sterilization in the sterilization cabinet 4. The filling may be performed in a manner that overfills each PCCS 42 slightly, where the runoff is collected and either reused or disposed of. By overfilling each PCCS 42 slightly, coverage of substantially the entire inner surface of each PCCS 42 can be ensured, such that the liquid hydrogen peroxide is able to sterilize substantially that entire inner surface of the PCCS 42.

The concentration of liquid H2O2 utilized may be any suitable concentration. According to some embodiments, the concentration of liquid H2O2 utilized in box 510 is greater than or equal to 3% and less than or equal to 90%. Examples of ranges of concentration of hydrogen peroxide that may be utilized in box 510 include, and are not limited to, 3-15%, 6-25%, 7-14%, and 25-36%. As another example of concentration of hydrogen peroxide, a concentration of 35%±1% may be used. Advantageously, the liquid H2O2 that is utilized in box 510 does not contain stabilizers. In some embodiments, citrate, acetate, phosphate, bicarbonate, or nitrate buffer stabilizer or stabilizers are acceptable for use to adjust or maintain a desired pH.

At box 520, the liquid H2O2 with which the PCCSs 42 were filled in box 510 is allowed to reside in each PCCS 42 for an exposure time. The exposure time required for sterilization is not instantaneous. That exposure time depends on factors such as load size, material inside the sterilization chamber 4, H2O2 concentration, temperature, and accelerants. Sterilization proceeds more quickly at higher H2O2 concentrations, at higher temperatures, and with the use of one or a combination of chemical accelerants such as anionic and/or non-ionic surfactants, phosphoric acid, and/or phosphonic acid. In traditional sterilization procedures using vapor hydrogen peroxide, the cycle time for sterilizing PCCSs 42 can be as long as eight hours or more, due to the need for deep vacuum in that process, and the need to remove all of the hydrogen peroxide from the PCCSs 42 so that remnant H2O2 does not attack the parenteral products with which the PCCSs 42 are later filled. The method 500 greatly reduces that cycle time for sterilizing PCCSs 42, and improves throughput for volume manufacturing of PCCSs 42 filled with parenteral products, by using pulsed UV light to neutralize any remnant H2O2 in the PCCSs 42, as described in greater detail below. Consequently, the exposure time of box 520 may be significantly reduced. As stated above, the specific exposure time depends on factors such as H2O2 concentration, temperature, and accelerants, which in turn depend in part on the PCCSs 42. For example, some PCCSs 42 may be fabricated from plastic or other material that is sensitive to high temperatures, and the exposure time is thus longer at a lower temperature. As another example, some PCCSs 42 may be fabricated from material that does not tolerate chemical accelerant as well as other material, and the exposure time is thus longer without the use of accelerant. As another example, some PCCSs 42 may be fabricated from material that does not tolerate high concentrations of H2O2 as well as lower concentrations, and the exposure time is thus longer at lower concentrations of H2O2.

At box 530, after the exposure time of box 520, the liquid hydrogen peroxide is removed from each PCCS 42, such as by gravity, by suction, or in any other appropriate manner. Where gravity is utilized, each PCCS 42 may be simply rotated to allow the liquid hydrogen to run out therefrom. In some applications or manufacturing processes, and for some PCCSs 42, such rotation may be advantageous. In other applications or manufacturing processes, and for other PCCSs 42, maintaining the PCCSs 42 in their original position and utilizing suction to remove the hydrogen peroxide may be advantageous. The liquid hydrogen peroxide may be removed from each PCCS 42 in any other suitable manner. At the end of box 530, a thin film of hydrogen peroxide may remain on the inner surface of each PCCS 42, and/or a small amount of hydrogen peroxide may remain in each PCCS 42.

Next, boxes 142-146 are performed as described above. Then, box 148 is performed as described above. At box 148, at least one UV light source 50, 54 is activated to generate UV light, and to illuminate the one or more PCCSs 42 in the sterilization position. According to some embodiments, pulsed UV light is generated at box 148. The UV light performs an additional function in the process of FIG. 20. Exposure of hydrogen peroxide to UV light causes that hydrogen peroxide to break down. That decomposition of H2O2 results in the formation of water (H2O) and oxygen (O2), both directly and through secondary reactions. Thus, not only does the exposure of the PCCSs 42 to the UV light cause sterilization of those PCCSs 42, but also any remaining H2O2 liquid remaining in each PCCS 42, whether that remaining H2O2 liquid is a film clinging to an interior of the PCCS 42, or is a small amount of liquid remaining at a lower point in the PCCS 42 is converted into water and oxygen by that UV light. The oxygen is in gaseous form and escapes each PCCS 42 to become part of the atmosphere. The remaining water in each PCCS 42 is a negligible amount. However, that negligible amount of water is consistent for each individual PCCS 42, as long as the previous boxes in the process are consistently performed, which can be controlled and monitored using process control tools. The amount of H2O2 with which each PCCS 42 is filled is always the same (within normal process variances), the method of removal of the H2O2 from the PCCS 42 prior to box 148 is always the same (within normal process variances), and as a result the quantity of water left in each PCCS 42 after box 148 is performed is always the same (within normal process variances). Using process control tools, it can be determined how much water will be left in a PCCS 42 after sterilization in box 148, such that the amount of parenteral product added to that PCCS 42 can be adjusted to avoid dilution by that negligible amount of water from the target concentration.

Boxes 150-156 are performed as described above. Optionally, at box 156, that quality control box also includes checking the level of water in a PCCS 42 to ensure that amount is within specification, if that amount of water is measurable. That water level may be checked in any suitable manner, such as by utilizing a scale to measure weight of the PCCS 42, a laser level transmitter, and/or an ultrasonic level transmitter, or by utilizing any other suitable liquid level measurement tools. If that level of water is acceptable, the process moves to box 158. If that level of water is not acceptable, the process moves to box 160.

Next, the sterilized PCCSs 42 are filled as described.

In some manufacturing processes and/or with particular PCCSs 42, it may be simpler and equally effective to perform method 500 by pre-sterilizing one or more PCCSs 42 with vapor-phase hydrogen peroxide instead of liquid hydrogen peroxide. The method 500 may be substantially the same, except for differences noted below. The vapor-phase hydrogen peroxide may be generated in any manner, such as by heating liquid hydrogen peroxide to substantially 120° C. Referring also to FIG. 20, at box 510, the PCCSs 42 are exposed to vapor-phase hydrogen peroxide, rather than being filled with liquid hydrogen peroxide. This exposure is performed before exposure of the PCCSs 42 to ultraviolet light. The concentration of hydrogen peroxide may be as described above with regard to liquid pre-sterilization.

At box 520, the vapor-phase hydrogen peroxide with which the PCCSs 42 were filled in box 510 is allowed to reside in each PCCS 42 for an exposure time. However, exposure of the PCCSs 42 to vapor-phase hydrogen peroxide a long time before their exposure to UV light may be advantageous in some situations. After being filled by vapor-phase hydrogen peroxide, some of the vapor-phase hydrogen peroxide will escape from the PCCSs 42, and the remainder will condense into liquid hydrogen peroxide, thereby continuing to kill microorganisms that may enter the PCCSs 42 over a long exposure time.

At box 530, after the exposure time of box 520, the vapor-phase hydrogen peroxide, and condensed liquid hydrogen peroxide, is removed from each PCCS 42, such as by gravity, by suction, or in any other appropriate manner. The exposure time may be selected such that a portion of the vapor-phase hydrogen peroxide wafts outward from each PCCS 42, and a portion condenses to liquid hydrogen peroxide inside each PCCS 42, such that only liquid hydrogen peroxide remains to be removed from each PCCS 42, and that liquid hydrogen peroxide may be removed from each PCCS 42 as described above. Suction may have particular utility in removing vapor-phase hydrogen peroxide from the PCCSs 42, because such suction may remove substantially all of the vapor-phase hydrogen peroxide that may remain in the PCCSs 42, as well as the condensed liquid hydrogen peroxide that may be present in the PCCSs.

The remainder of the process of FIG. 20 may then proceed as described above.

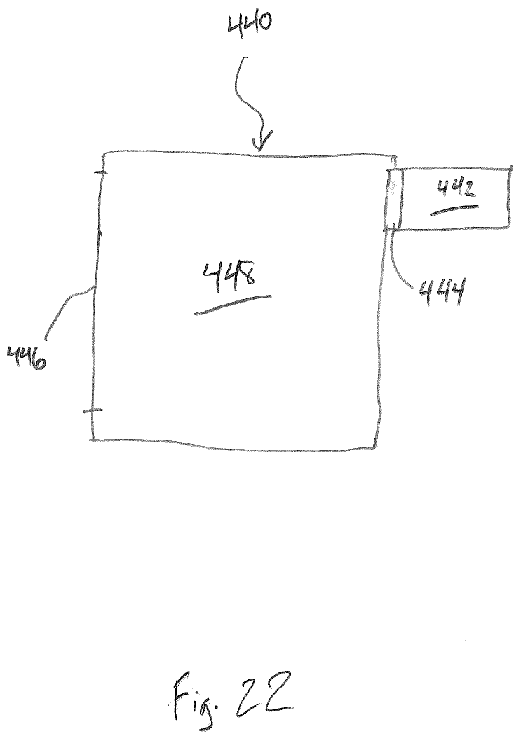

Referring also to FIG. 22, pulsed or continuous UV light may be used to enhance sterilization of PCCSs 42 in a traditional hydrogen peroxide sterilization process. Referring to the process 400 shown in FIG. 21, at box 402 one or more PCCSs 42 are loaded into an interior 448 of a sterilization chamber 440. The sterilization chamber 440 may be simply a scalable volume capable of holding one or more PCCSs 42, and capable of maintaining interior conditions suitable for liquid or vapor phase hydrogen peroxide sterilization. A door 446 provides access to an interior 448 of the chamber 440, and is scalable to prevent escape of hydrogen peroxide and UV light. A UV light source 442 may be positioned relative to the chamber 440 in order to illuminate the interior 448 of the chamber 440. According to some embodiments, the UV light source 442 may be positioned within the chamber 442. According to other embodiments, the chamber 440 may include a window 444 substantially transparent to UV light, and the UV light source 442 may be positioned outside the chamber 400 relative to the window 444 such that the UV light source 442 is configured to illuminate the interior of the chamber 400 at an appropriate point or points in the process 400. The UV light source 442 may be the same light source as, or substantially similar to, the UV light sources 50, 54 described above.

Next, at box 404, preconditioning is performed on the interior 448 of the chamber 440. During preconditioning, a vacuum is applied to the interior 448 of the chamber 440 holding the PCCSs 42, and as a result humidity in the interior 448 of the chamber 440 is driven to an optimal level. The vacuum may be in the range of 1-10 millibar (0.03-0.3 inches Hg). According to other embodiments, the vacuum applied to the interior 448 of the chamber 440 may be higher or lower than that range. The preconditioning performed at box 402 may last between one to two hours. According to other embodiments, the preconditioning performed at box 402 may last less than one hour, or longer than two hours.

Next, at box 406, the PCCSs 42 in the interior 448 of the chamber 440 are sterilized. During sterilization in box 406, vaporized hydrogen peroxide is injected into the interior 448 of the chamber 440 in pulses. Each pulse allows for injection of fresh vapor phase hydrogen peroxide into the interior 448 of the chamber 440, after at least part of the vapor phase hydrogen peroxide in the previous pulse has been consumed in the process of encountering contaminants and dissociated. In some embodiment, 35% liquid H2O2 is vaporized at over 100° C. and the vapor is injected into the interior 448 of the chamber 440 in the form of pulses to reach a concentration in the chamber 440 of 1-2 mg/L. According to other embodiments, a different concentration of liquid H2O2 is vaporized. According to other embodiments, the concentration in the interior 448 of the chamber 440 may be less than 1 mg/L, or greater than 2 mg/L.

Next, at box 408, in the aeration phase residual H2O2 is removed from the interior 448 of the chamber 440 and the PCCSs 42 in the chamber 440. That remnant H2O2 may be in the form of a film on the PCCSs 42, liquid at the bottom of PCCSs 42, or other vapor phase or condensed H2O2 in or on the PCCSs 42. In the prior art, this was performed by applying vacuum to the interior 448 of the chamber 440 and injecting air or steam to remove any residual H2O2 from the PCCSs 42 in the interior 448 of the chamber 440. However, that process could last from two to five hours in the prior art. In an aspect of the present invention, at box 408, the PCCSs 42 in the interior 448 of the chamber 440 are exposed to pulsed UV light, as described above at box 148 of FIG. 9. Exposure to UV light causes remnant H2O2 to decompose into hydroxyl radicals, which in turn combine to form end products of water (H2O) and oxygen (O2). That exposure time may be on the order of 50-90 minutes, depending on the power output of the UV light. The higher the power output, the less time is required to decompose remnant H2O2. In combination with the typical aeration step in the VHP process, the illumination of the PCCSs 42 in the interior 448 of the chamber 440 with UV light thus reduces the time spent in box 408 to 50-90 minutes, compared to two to five hours in the prior art. According to other embodiments, the PCCSs 42 in the interior 448 of the chamber 440 may be exposed to continuous UV light across a period of time at box 408. Such continuous UV light may be at a lower power output than a pulsed UV light, but may run for a period of time that allows that UV light to dissociate remnant H2O2 molecules. The use of such continuous UV light still reduces the time required for performing box 408 compared to the prior art, but may not reduce that time as much as a higher-powered pulsed UV light. Integrating the curve of power across time (where time is measured on the x-axis) provides a useful measure of total power applied to remnant H2O2 in and on the PCCSs 42 in the interior 448 of the chamber 440, and a continuous UV light source may deliver total power over a similar or longer period of exposure time as a pulsed UV light source. The use of pulsed or continuous UV light to decompose remnant H2O2 also allows for higher concentrations of vapor phase H2O2 to be used compared to the prior art, because the UV light breaks down that higher concentration H2O2 while still reducing aeration time after exposure to H2O2 at higher concentrations compared to the prior art. Acceleration of the aeration phase at box 408 accelerates the entire sterilization process, and may allow a manufacturing facility to operate two batches or more per day per chamber 440, as opposed to a single batch per day with the aeration process of the prior art. It is noted that the exposure time during the performance of box 408 may be less than the exposure time during the performance of box 148 of FIG. 9, because the process of FIG. 21 relies on both UV light and hydrogen peroxide for sterilization, and the process of FIG. 20 relies only on UV light.

Next, at box 410, the PCCSs 42 are removed from the interior 448 of the chamber 440, and filled in any suitable manner.

The process 400 described above has been described as a VHP process. However, it will be apparent to one skilled in the art that the use of pulsed UV light is equally effective when used to enhance a liquid hydrogen peroxide sterilization process. Indeed, the use of pulsed UV light may be even more advantageous where liquid H2O2 is used, because sterilization with liquid H2O2 requires a greater quantity thereof, and thus may leave behind a greater quantity of remnant liquid H2O2 that must be removed from the PCCSs 42 before filling.

### Sterilization and/or Sanitization of Other Containers or Items

The process 400 above has been described as useful for sterilizing PCCSs 42. It will be appreciated by one skilled in the art that the process 400 also may find use in sterilizing or sanitizing containers, in other fields of endeavor. As one example, the process 400 may be used to sterilize or sanitize labware, such as but not limited to beakers, pipettes, graduated cylinders and petri dishes. As another example, the process 400 may be used to sterilize or sanitize diagnostic equipment and/or reusable items utilized in conjunction with such diagnostic equipment. As another example, the process 400 may be used to sterilize or sanitize containers to be filled with cosmetics or perfume. As another example, the process 400 may be used to sterilize or sanitize bottles or other containers for beverages, whether alcoholic or non-alcoholic. As another example, the process 400 may be used to sterilize or sanitize bottles, cans, jars, or other containers for food items. Sterilization or sanitization of such containers may be performed as described above with regard to the process 400. Where sterilization is not required and sanitization is sufficient, the concentration and duration of H2O2 applied to the containers may be reduced, and consequently the concentration and duration of UV light applied to such containers after their exposure to H2O2 may be reduced as well,, allowing such containers to be sanitized in less time overall than would be required for their sterilization.

In addition, it will be appreciated by one skilled in the art that the process 400 may be useful for sterilizing or sanitizing items that are not containers, in other fields of endeavor. As one example, the process 400 may be used to sterilize medical devices. Such medical devices may include catheters or other tubing. Items that include tubing are often challenging to sterilize, and as a result ethylene oxide is often used to sterilize them. However, ethylene oxide is being phased out by federal regulation. Vapor-phase H2O2 combined with UV light allows for vapor-phase H2O2 to enter and sterilize the lumen of such tubing, and the application of UV light to such tubing afterwards converts remnant H2O2 to sterile water and oxygen, providing sterilization without the use of ethylene oxide. Such a process would be particularly effective where the tubing is clear or translucent. As another example, the process 400 may be used to sterilize medical devices, whether implantable or not, that do not include tubing, such as but not limited to stents, knee implants, and hernial mesh. Liquid or vapor phase H2O2 may be used to sterilize the surface of such items, and the application of UV light to such items afterwards converts remnant H2O2 on the surface of such items into sterile water and oxygen. As another example, the process 400 may be used to sterilize medical, dental, or veterinary tools that are not implantable, such as forceps, scalpels, and picks. Liquid or vapor phase H2O2 may be used to sterilize the surface of such items, and the application of UV light to such items afterwards converts remnant H2O2 on the surface of such items into sterile water and oxygen.

As used in this document, and as customarily used in the art, terms of approximation, including the words “substantially” and “about,” are defined to mean normal variations in the dimensions, measurements and physical properties of items and processes in the physical world that may be associated with accuracy, precision, and/or tolerances.

While the invention has been described in detail, it will be apparent to one skilled in the art that various changes and modifications can be made and equivalents employed, without departing from the present invention. It is to be understood that the invention is not limited to the details of construction, the arrangements of components, and/or the method set forth in the above description or illustrated in the drawings. Statements in the abstract of this document, and any summary statements in this document, are merely exemplary; they are not, and cannot be interpreted as, limiting the scope of the claims. Further, the figures are merely exemplary and not limiting. Topical headings and subheadings are for the convenience of the reader only. They should not and cannot be construed to have any substantive significance, meaning or interpretation, and should not and cannot be deemed to indicate that all of the information relating to any particular topic is to be found under or limited to any particular heading or subheading. Therefore, the invention is not to be restricted or limited except in accordance with the following claims and their legal equivalents.

What is claimed is:

1. A method for sterilizing workpiece items, comprising:
providing a sterilization cabinet, comprising
a sterilization chamber,
at least one portal to said sterilization chamber openable to load at least one workpiece item into said sterilization chamber,
a carrier movable within said sterilization chamber, and
at least one UV light source that generates pulsed UV light pulses directed into said sterilization chamber;
exposing at least one workpiece item to hydrogen peroxide;
after said exposing, loading at least one workpiece item onto a carrier in said sterilization chamber, said carrier in a loading position;
moving said carrier to move at least one workpiece item to a sterilization position within said sterilization chamber;
sterilizing each workpiece item by applying pulsed UV light thereto;
moving said carrier to an unloading position; and
causing each workpiece item to exit the sterilization chamber.

2. The method of claim 1, wherein, during said exposing, said hydrogen peroxide is in liquid phase.

3. The method of claim 1, wherein, during said exposing, said hydrogen peroxide is in vapor phase.

4. The method of claim 1, wherein said exposing is performed for an exposure time sufficiently long to sterilize the at least one workpiece item.

5. The method of claim 1, wherein the at least one workpiece item is a PCCS; further comprising filling each at least one workpiece item with a parenteral product after said causing.

6. The method of claim 5, further comprising removing each PCCS from said carrier after said filling.

7. The method of claim 1, further comprising storing the at least one workpiece item between said exposing and said loading.

8. A method for sterilizing at least one workpiece item, comprising:
providing a sterilization chamber configured for hydrogen peroxide sterilization and a UV light source configured to illuminate the interior of said sterilization chamber;
loading the at least one workpiece item into said sterilization chamber;
contacting the at least one workpiece item with hydrogen peroxide;
separating the at least one workpiece item from said hydrogen peroxide, leaving remnant hydrogen peroxide on the at least one workpiece item; and
illuminating the at least one workpiece item with UV light, wherein said illuminating decomposes said remnant hydrogen peroxide on the at least one workpiece item.

9. The method of claim 8, wherein said illuminating comprises illuminating the at least one workpiece item with pulsed UV light.

10. The method of claim 8, wherein said illuminating comprises illuminating the at least one workpiece item with continuous UV light.

11. The method of claim 8, wherein said contacting comprises contacting the at least one workpiece item with vapor phase hydrogen peroxide using a three stage process of preconditioning, sterilization, and aeration; and wherein said illuminating is performed during said aeration.

12. The method of claim 8, wherein said contacting comprises contacting the at least one workpiece item with liquid hydrogen peroxide.

13. The method of claim 8, further comprising filling each at least one workpiece item with a parenteral product after said illuminating.

14. A method for sterilizing at least one workpiece, comprising:
exposing the at least one workpiece to a hydrogen peroxide environment for an exposure time of sufficient duration to sterilize the at least one workpiece;
separating the at least one workpiece from said hydrogen peroxide environment; and
illuminating the at least one workpiece with ultraviolet light to decompose remnant hydrogen peroxide to water and oxygen.

15. The method of claim 14, wherein hydrogen peroxide environment is a liquid phase hydrogen peroxide environment.

16. The method of claim 14, wherein hydrogen peroxide environment is a vapor phase hydrogen peroxide environment.

17. The method of claim 16, wherein said hydrogen peroxide environment is an interior of a chamber filled with said vapor phase hydrogen peroxide, and wherein separating comprises applying a vacuum to said interior of said chamber.

18. The method of claim 14, wherein said illuminating is performed with pulsed UV light.

19. The method of claim 14, wherein said illuminating is performed with continuous UV light.

20. The method of claim 15, wherein at least part of said separating is performed at the same time as at least part of said separating.
